(19) United States
(12) Patent Application Publication
(10) Pub. No.: US 2022/0213126 A1
(43) Pub. Date: Jul. 7, 2022

(54) SMALL MOLECULE AUTOPHAGY INDUCERS FOR THE TREATMENT OF CANCER AND NEURODEGENERATIVE DISEASES

(71) Applicant: John Wayne Cancer Institute, Santa Monica, CA (US)

(72) Inventors: Venkata Yenugonda, Woodland Hills, CA (US); Santosh Kesari, Santa Monica, CA (US)

(73) Assignee: John Wayne Cancer Institute, Santa Monica, CA (US)

(21) Appl. No.: 17/605,362

(22) PCT Filed: Apr. 23, 2020
(86) PCT No.: PCT/US2020/029580

§ 371 Date: Oct. 21, 2021

Related U.S. Application Data
(60) Provisional application No. 62/838,001, filed on Apr. 24, 2019.

(51) Int. Cl. C07F 5/02; A61K 45/06; A61P 35/04

(52) U.S. Cl. CPC C07F 5/025; A61P 35/04; A61K 45/06

(57) ABSTRACT

Disclosed herein are compounds and methods for treating cancer and neurodegenerative diseases. In some examples, the compounds increase autophagy.

### CROSS REFERENCE TO RELATED APPLICATIONS

This application claims the benefit of U.S. Provisional Application No. 62/838,001, filed Apr. 24, 2019, which is incorporated herein by reference in its entirety.

### FIELD

This disclosure relates to autophagy-stimulating compositions and methods of their use, particularly for treating cancer and neurodegenerative disease.

### BACKGROUND

Triple negative breast cancer (TNBC) affects a significant portion of women in the United States, with a high frequency of recurrence, metastatic disease, and short survival times. Mortality rates are highest among African-American women and limited treatment options are available. The absence of hormone and Her2 receptors on TNBC precludes the use of targeted therapies that are available for other breast cancer subtypes, and cytotoxic chemotherapy such as paclitaxel and doxorubicin remains the mainstay of treatment for TNBC.

Glioblastoma multiforme (GBM) is one of the most aggressive primary brain tumors, with a poor prognosis despite maximal standard of care treatment. The overall survival rate for patients with this disease has not significantly increased, despite decades of research. Recurrence of GBM is common, and its management is often unclear and case-dependent.

Protein aggregation is the main cause of several human neurodegenerative diseases such as Alzheimer's disease (AD), Parkinson's disease (PD), Huntington's disease (HD), and amyotrophic lateral sclerosis (ALS). The aggregates usually consist of fibers containing misfolded protein with a β-sheet conformation, termed amyloid. There is partial but not perfect overlap among the cells in which abnormal proteins are deposited and the cells that degenerate. Protein aggregation is a highly complex process resulting in formation of a variety of aggregates with different structures and morphologies. Many of them are highly cytotoxic. The clearance of these misfolded proteins may represent a promising therapeutic strategy in these diseases.

### SUMMARY

New treatments for cancers (such as TNBC and GBM) and neurodegenerative diseases are needed. Disclosed herein are methods of treating of TNBC and GBM with VMY-BC-1. Also disclosed are compounds and methods of treatment of cancers and neurodegenerative diseases.

In some embodiments, the disclosure includes methods of treating triple-negative breast cancer, brain cancer, or a neurodegenerative disorder in a subject, comprising administering to the subject an effective amount of a composition comprising Formula I:

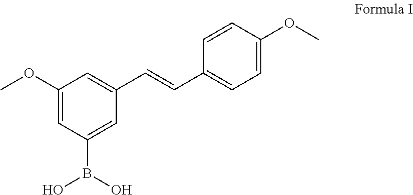

or a pharmaceutically acceptable salt, stereoisomer, tautomer, or prodrug thereof, thereby treating the triple-negative breast cancer, brain cancer (such as glioblastoma), or neurodegenerative disorder (such as Alzheimer's disease, Parkinson's disease, Huntington's disease, or amyotrophic lateral sclerosis).

Also disclosed herein are embodiments of a compound having a structure of Formula II, IIA, or IIB:

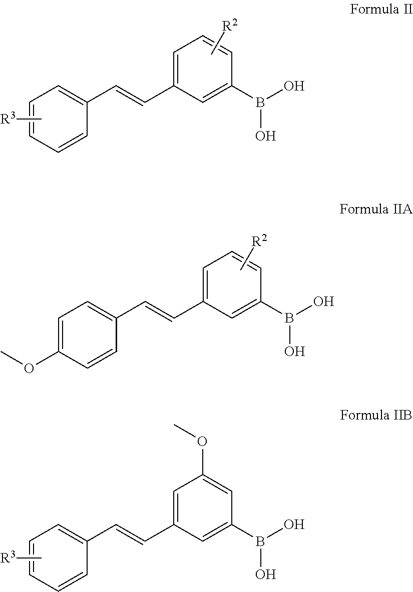

or a pharmaceutically acceptable salt, stereoisomer, tautomer, or prodrug thereof, wherein each of R² and R³ independently are selected from aliphatic, haloaliphatic, halogen, —C(O)OR^{b}, —C(O)N(R^{b})₂, —C(O)H, —OC(O)R^{b}, —OC(O)OR^{b}, —NR^{b}S(O)₂R^{b}, —S(O)₂N(R^{b})₂, —[(C(R^{b})₂)ₙO]ₘR^{b}, —OR^{b}, —SR^{b}, —N(R^{b})₂, wherein each R^{b} independently is hydrogen, aliphatic, heteroaliphatic, haloaliphatic, aromatic, or combinations thereof; and each of n and m independently is an integer ranging from 1 to 50.

In any or all of the above embodiments, each R^{b} independently is hydrogen, alkyl, alkenyl, alkynyl, heteroalkyl, heteroalkenyl, heteroalkynyl, haloalkyl, haloalkenyl, haloalkynyl, aryl, heteroaryl, or combinations thereof.

In any or all of the above embodiments, each of R² and R³ independently is selected from alkyl, —C(H)(halogen)₂, —C(H₂)(halogen), —C(halogen)₃, Cl, F, Br, I, —C(O)OH, —C(O)NH₂, —C(O)N(H)alkyl, —C(O)N(alkyl)₂, —C(O)O-alkyl, —OC(O)-alkyl, —OC(O)O-alkyl, —NHS(O)₂alkyl, —N(alkyl)S(O)₂alkyl, —S(O)₂NH₂, —S(O)₂N(alkyl)₂, —S(O)₂N(H)alkyl, [—(CH₂)˜O-]ₘH, —OH, O-alkyl, —O-heteroalkyl, —SH, —S-alkyl, —S-heteroalkyl, —NH₂, —N(alkyl)₂, or —N(H)alkyl, wherein each alkyl group independently is selected from lower alkyl and wherein the heteroalkyl group is —(CH₂)_{q}N(alkyl)₂, wherein q is an integer selected from 1, 2, or 3.

In any or all of the above embodiments, each of R² and R³ independently is selected from methyl, ethyl, propyl, isopropyl, butyl, isobutyl, t-butyl, Cl, F, —CF₃, —COOH, —OC(O)Me, —C(O)NH₂, NH₂, —NMe₂, —NHMe, —SO₂NH₂, —OEt, —O(CH₂)₂N(Me)₂, or —OiPr.

Also disclosed herein are embodiments of a compound having a structure of Formula III, IIIA, IIIB, or IIIC

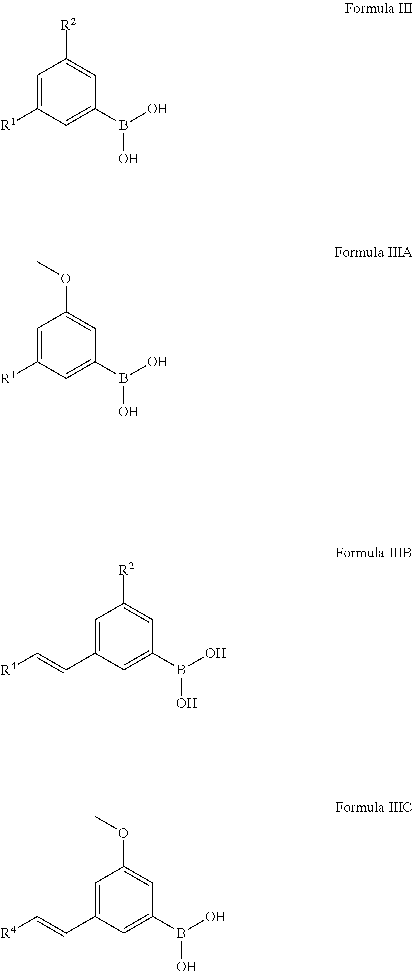

or a pharmaceutically acceptable salt, stereoisomer, tautomer, or prodrug thereof, wherein R¹ is —[(CH═CH)-]ₚheterocyclic, —[(CH═CH)-]ₚaromatic, or —[(CH═CH)-]ₚheterocyclic-aromatic, wherein p is 0 or 1; R² is selected from aliphatic, haloaliphatic, halogen, —C(O)OR^{b}, —C(O)N(R^{b})₂, —C(O)H, —OC(O)R^{b}, —OC(O)OR^{b}, —NR^{b}S(O)₂R^{b}, —S(O)₂N(R^{b})₂, —[(C(R^{b})₂)ₙO]ₘR^{b}, —OR^{b}, —SR^{b}, —N(R^{b})₂, wherein each R^{b} independently is hydrogen, aliphatic, heteroaliphatic, haloaliphatic, aromatic, or combinations thereof, wherein each of n and m independently is an integer ranging from 1 to 50; and R⁴ is naphthyl, pyridinyl, pyrrole, furanyl, thiophenyl, quinolinyl, piperidinyl, azepanyl, or diazabicyclooctanyl.

In any or all of the above embodiments, R¹ is —[(CH═CH)-]ₚheterocyclic or —[(CH═CH)-]ₚheterocyclic-aromatic and wherein the heterocyclic group comprises 2 to 10 carbon atoms and one or more heteroatoms selected from oxygen, nitrogen, or combinations thereof.

In any or all of the above embodiments, R¹ is —[(CH═CH)-]ₚaromatic or —[(CH═CH)-]ₚheterocyclic-aromatic and the aromatic group is an aryl group or a heteroaryl group.

In any or all of the above embodiments, R¹ is —[(CH═CH)-]ₚheterocyclic-aromatic and the heterocyclic-aromatic group comprises one or more heterocyclic groups fused with one or more aromatic groups.

In any or all of the above embodiments, p is 0 and R¹ is carbazolyl, dihydrobenzodioxinyl, dibenzofuranyl, or xanthenyl.

In any or all of the above embodiments, p is 0, wherein the compound is any one of:

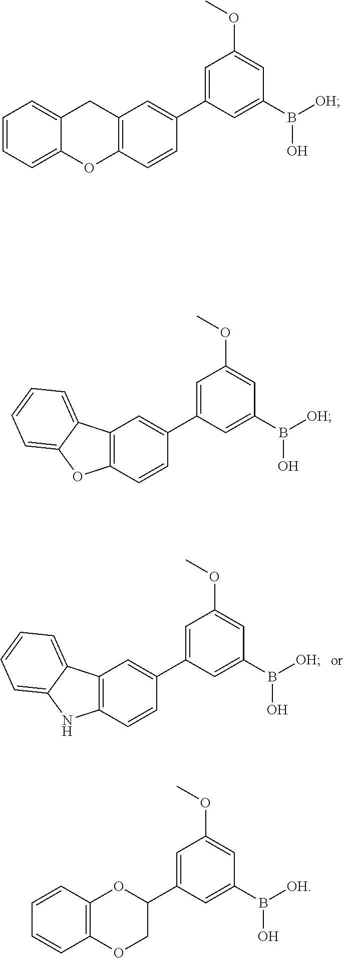 [SMILES: COc1cc(B(O)O)cc(-c2ccc3c(c2)Cc2ccccc2O3)c1 ; COc1cc(B(O)O)cc(-c2ccc3oc4ccccc4c3c2)c1 ; COc1cc(B(O)O)cc(-c2ccc3[nH]c4ccccc4c3c2)c1 ; COc1cc(B(O)O)cc(C2COc3ccccc3O2)c1]

In any or all of the above embodiments, R¹ is —[(CH═CH)-]ₚheterocyclic, —[(CH═CH)-]ₚaromatic, or —[(CH═CH)-]ₚheterocyclic-aromatic, wherein p is 1.

In any or all of the above embodiments, the compound has a structure of Formula IIIB or IIIC:

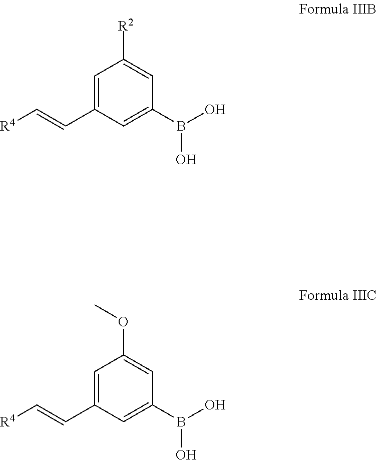

wherein R² is isopropyl, —NMe₂, —OEt, —OPr, —OBu, —O(CH₂)₂N(Me)₂, or —OiPr; and R⁴ is naphthyl, pyridinyl, pyrrole, furanyl, or thiophenyl.

Also disclosed herein are embodiments of a compound having a structure of Formula IV:

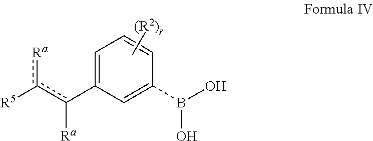

or a pharmaceutically acceptable salt, stereoisomer, tautomer, or prodrug thereof, wherein each R^{a} independently is hydrogen, halogen, aliphatic, heteroaliphatic, haloaliphatic, aromatic, or any combination thereof; R² is selected from aliphatic, haloaliphatic, halogen, —C(O)OR^{b}, —C(O)N(R^{b})₂, —C(O)H, —OC(O)R^{b}, —OC(O)OR^{b}, —NR^{b}S(O)₂R^{b}, —S(O)₂N(R^{b})₂, [(C(R^{b})₂)ₙO]ₘR^{b}, —OR^{b}, SR^{b}, —N(R^{b})₂, wherein each R^{b} independently is hydrogen, aliphatic, heteroaliphatic, haloaliphatic, aromatic, or combinations thereof, wherein each of n and m independently is an integer ranging from 1 to 50; and R⁵ is either selected from naphthyl, pyridinyl, pyrrole, furanyl, thiophenyl, quinolinyl, piperidinyl, azepanyl, or diazabicyclooctanyl, or is an aromatic group comprising an R³ substituent, wherein the R³ group is selected from aliphatic, haloaliphatic, halogen, —C(O)OR^{b}, —C(O)N(R^{b})₂, —C(O)H, —OC(O)R^{b}, —OC(O)OR^{b}, —NR^{b}S(O)₂R^{b}, —S(O)₂N(R^{b})₂, —[(C(R^{b})₂)ₙO]ₘR^{b}, —OR^{b}, —SR^{b}, —N(R^{b})₂, wherein each R^{b} independently is hydrogen, aliphatic, heteroaliphatic, haloaliphatic, aromatic, or combinations thereof, wherein each of n and m independently is an integer ranging from 1 to 50; and r is an integer selected from 0 or 1.

In some embodiments, the compound further has a structure of Formula IVA

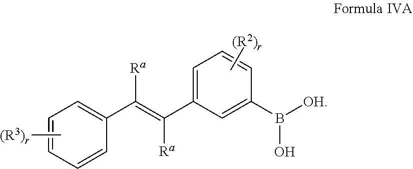

In some embodiments, the compound further has a structure of Formulas IVB, IVC, IVD, IVE, or IVF.

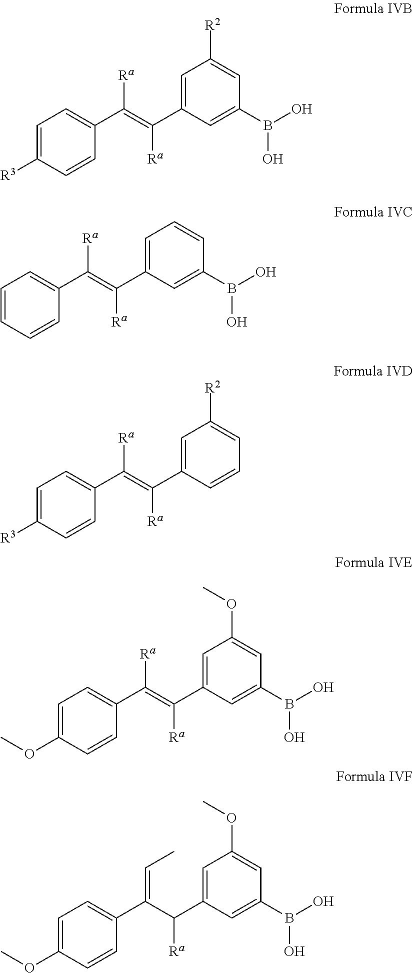

In any or all of the above embodiments, each R^{a} independently is hydrogen, halogen, aliphatic, heteroaliphatic, haloaliphatic, aromatic, or any combination thereof.

In any or all of the above embodiments, each R^{a} independently is halogen, aliphatic, aromatic, or any combination thereof.

In any or all of the above embodiments, each R^{a} independently is hydrogen, F, Br, Cl, I, or alkyl.

In any or all of the above embodiments, each R^{a} independently is F, ethyl, phenyl, or -PhO(CH₂)₂NMe₂.

In any or all of the above embodiments, each R^{a} is different.

Also disclosed are methods of treating a subject with cancer or a neurodegenerative disease, comprising administering an effective amount of the compound of any one of Formulas II-IV to the subject. In some embodiments, the subject with cancer has a solid tumor (for example, breast cancer or glioblastoma) or a hematological malignancy. In other embodiments, the subject has a neurodegenerative disease (for example, Alzheimer's disease, Parkinson's disease, Huntington's disease, or amyotrophic lateral sclerosis).

The foregoing and other features of the disclosure will become more apparent from the following detailed description, which proceeds with reference to the accompanying figures.

### BRIEF DESCRIPTION OF THE DRAWINGS

FIGS. 1A-1C are the structures of VMY-BC-1 (FIG. 1A), resveratrol (FIG. 1B), and LYN-1604 (FIG. 1C).

FIGS. 2A and 2B are graphs showing the effect of VMY-BC-1 on TNBC cells. FIG. 2A shows the effect of VMY-BC-1 on MDA-MB-231 cells with different amounts of serum (1%, 5%, or 10%) in the medium. FIG. 2B shows MDA-MB-231 cells incubated for 24-72 hours in Transwells coated with Matrigel and treated with 10 μM VMY-BC-1, 10 μM bosutinib, or chemoattractant (10% FBS). The percentage of cells crossing the chamber is presented as the mean±SEM. *P<0.05.

FIG. 3 is a schematic diagram showing proteomic profiling of MDA-MB-231 TNBC cells treated with VMY-BC-1 using quantitative multiplexed tandem tagged isotope labeling.

FIG. 4 is a heatmap of Ingenuity pathway analysis of quantified proteins showing top pathway activities increased (red) or decreased (green) by VMY-BC-1. Z scores with P values <0.05 were significant.

FIGS. 5A and 5B show VMY-BC-1 pathway analysis. FIG. 5A is a schematic of a cellular thermal shift assay for VMY-BC-1 target identification in MDA-MB-1 cells and FIG. 5B shows results from the assay.

FIG. 6 is a Western blot of autophagy proteins in TNBC cells treated with vehicle (control), VMY-BC-1, or Rapamycin. Western blot is shown for p-ULK1, LC3 A/B, and cleaved PARP.

FIGS. 7A-7C show a docking model of VMY-BC-1 with ULK1. FIG. 7A: VMY-BC-1 docking in the agonist pocket. FIG. 7B: Covalent bond between VMY-BC-1 and serine residue 87 (S87) in ULK1 kinase domain. FIG. 7C: Overlay of LYN-1604 (yellow) and VMY-BC-1 (green) in ULK1 agonist site.

FIG. 8 is a graph showing plasma pharmacokinetics of VMY-BC-1 in vivo. Concentrations are expressed as the mean±SEM.

FIGS. 9A and 9B are graphs showing tumor volume (FIG. 9A) and body weight (FIG. 9B) in MDA-MB-231 xenograft mice administered 75 mg/kg VMY-BC-1 daily for 14 days. Data are expressed as the mean±SEM. P=0.0001.

FIGS. 10A and 10B are graphs showing plasma (FIG. 10A) and tumor (FIG. 10B) concentrations of VMY-BC-1 in MDA-MB-231 xenograft mice administered 75 mg/kg VMY-BC-1.

FIG. 11 is a series of panels showing number of Ki67-positive cells in vehicle- and VMY-BC-1-treated tumors (top), representative immunohistochemistry images (middle), and data quantitation (bottom).

FIG. 12 is a series of panels showing number of TUNEL-positive cells in vehicle- and VMY-BC-1-treated tumors (top), representative TUNEL staining images (middle), and data quantitation (bottom).

FIG. 13 is a Western blot of autophagy proteins in TNBC xenograft. Tumor tissue from mice bearing MDA-MB-231 tumors was extracted 24 hours after treatment with vehicle control or VMY-BC-1.

FIG. 14 is a Western blot of autophagy proteins in U87 GBM cells treated with vehicle (control), VMY-BC-1, or Rapamycin. Western blot is shown for p-ULK1, LC3 A/B, and cleaved PARP.

FIG. 15 is a graph showing the effect of 5 μM or 10 μM VMY-BC-1 on U251 GBM cell cycle over time.

FIGS. 16A and 16B are graphs showing plasma (FIG. 16A) and brain (FIG. 16B) concentrations of VMY-BC-1 in mice administered 25 mg/kg VMY-BC-1.

FIGS. 17A and 17B are graphs showing tumor volume (FIG. 17A) and body weight (FIG. 17B) in U87 xenograft mice administered 75 mg/kg VMY-BC-1 daily for 14 days. Data are expressed as the mean±SEM. P=0.0001.

FIGS. 18A-18C are graphs showing plasma (FIG. 18A) and tumor (FIG. 18B) concentrations of VMY-BC-1 in U87 xenograft mice administered 75 mg/kg VMY-BC-1. FIG. 18C shows a summary of concentrations in plasma and tumor over time.

FIG. 19 is a series of panels showing number of Ki67-positive cells in vehicle- and VMY-BC-1-treated U87 tumors (top), representative immunohistochemistry images (middle), and data quantitation (bottom).

FIG. 20 is a series of panels showing number of TUNEL-positive cells in vehicle- and VMY-BC-1-treated U87 tumors (top), representative TUNEL staining images (middle), and data quantitation (bottom).

FIGS. 21A and 21B show the effect of VMY-BC-1 on polyglutamine aggregations (FIG. 21A) and lifespan (FIG. 21B) in C. elegans.

FIG. 22 shows an exemplary synthetic scheme for VMY-BC-1.

### DETAILED DESCRIPTION

Autophagy is a conserved catabolic process that maintains homeostasis by regulating the energy balance of the cell. Depending on tumor type and environment, autophagy modulation plays an important role in tumor cell survival and inhibition pathways. Increasing evidence supports that chemical modulation of autophagy inhibition and activation holds a therapeutic potential and these studies have led to the initiation of multiple clinical trials combining chemotherapeutic agents and autophagy inhibitors and activators for various cancer types. Nonspecific autophagy inhibitors/activators have been widely used in a number of clinical trials, including GBM, as an adjuvant therapy with chemotherapeutic agents. However, lack of specificity of these compounds is associated with toxicity that diminishes efficacy.

Protein aggregation is associated with human neurodegenerative diseases (including AD, PD, HD, and ALS). The two main routes for intracellular protein degradation are the ubiquitin-proteasome and the autophagy-lysosome pathways.

The compound VMY-BC-1 is predicted to bind to ULK1, and is shown herein to stimulate apoptosis and autophagy pathways and increase phosphorylation of autophagy proteins in tumor cells. As disclosed herein, the autophagy activator VMY-BC-1 inhibited growth of triple-negative breast cancer cells and glioblastoma cells both in vitro and in vivo. In addition, VMY-BC-1 was shown to reduce poly-glutamine aggregates in C. elegans at low dose. These results suggest that the disclosed compositions can be useful in treating cancer and neurodegenerative diseases.

### I. Terms

To facilitate review of the various embodiments of the disclosure, the following explanations of specific terms are provided. Certain functional group terms include a symbol “-” which is used to show how the defined functional group attaches to, or within, the compound to which it is bound. Also, a dashed bond (e.g., “--”) as used in certain formulas described herein indicates an optional bond (that is, a bond that may or may not be present). A dashed bond in combination with a single bond (e.g., “ [symbol] ”) as used in certain formulas described herein indicates an optional double bond (that is, the bond can be a double bond and, if not, then it is a single bond). A wavy bond (e.g., “ [symbol] ”) indicates a point of disconnection. A person of ordinary skill in the art would recognize that the definitions provided below and the compounds and formulas included herein are not intended to include impermissible substitution patterns (e.g., methyl substituted with 5 different groups, and the like). Such impermissible substitution patterns are easily recognized by a person of ordinary skill in the art. In formulas and compounds disclosed herein, a hydrogen atom is present and completes any formal valency requirements (but may not necessarily be illustrated) wherever a functional group or other atom is not illustrated. For example, a phenyl ring that is drawn as

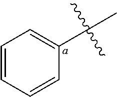 [SMILES: *c1ccccc1]

comprises a hydrogen atom attached to each carbon atom of the phenyl ring other than the “a” carbon, even though such hydrogen atoms are not illustrated. Any functional group disclosed herein and/or defined above can be substituted or unsubstituted, unless otherwise indicated herein.

Unless otherwise explained, all technical and scientific terms used herein have the same meaning as commonly understood by one of ordinary skill in the art to which this disclosure belongs. The singular terms “a,” “an,” and “the” include plural referents unless the context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

Definitions of common terms in chemistry may be found in Richard J. Lewis, Sr. (ed.), Hawley's Condensed Chemical Dictionary, published by John Wiley & Sons, Inc., 2016 (ISBN 978-1-118-13515-0). Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar references.

In order to facilitate review of the various embodiments of the disclosure, the following explanations of specific terms are provided:

Aldehyde: —C(O)H.

Aliphatic: A hydrocarbon group having at least one carbon atom to 50 carbon atoms (C₁₋₅₀), such as one to 25 carbon atoms (C₁₋₂₅), or one to ten carbon atoms (C₁₋₁₀), and which includes alkanes (or alkyl), alkenes (or alkenyl), alkynes (or alkynyl), including cyclic versions thereof, and further including straight- and branched-chain arrangements, and all stereo and position isomers as well.

Alkenyl: An unsaturated monovalent hydrocarbon having at least two carbon atom to 50 carbon atoms (C₂₋₅₀), such as two to 25 carbon atoms (C₂₋₂₅), or two to ten carbon atoms (C₂₋₁₀), and at least one carbon-carbon double bond, wherein the unsaturated monovalent hydrocarbon can be derived from removing one hydrogen atom from one carbon atom of a parent alkene. An alkenyl group can be branched, straight-chain, cyclic (e.g., cycloalkenyl), cis, or trans (e.g., E or Z).

Alkyl: A saturated monovalent hydrocarbon having at least one carbon atom to 50 carbon atoms (C₁₋₅₀), such as one to 25 carbon atoms (C₁₋₂₅), or one to ten carbon atoms (C₁₋₁₀), wherein the saturated monovalent hydrocarbon can be derived from removing one hydrogen atom from one carbon atom of a parent compound (e.g., alkane). An alkyl group can be branched, straight-chain, or cyclic (e.g., cycloalkyl).

Alkynyl: An unsaturated monovalent hydrocarbon having at least two carbon atom to 50 carbon atoms (C₂₋₅₀), such as two to 25 carbon atoms (C₂₋₂₅), or two to ten carbon atoms (C₂₋₁₀), and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (e.g., cycloalkynyl).

Aromatic: A cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized TE-electron system. Typically, the number of out of plane π-electrons corresponds to the Hückel rule (4n+2). The point of attachment to the parent structure typically is through an aromatic portion of the condensed ring system. For example,

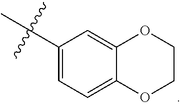 [SMILES: *c1ccc2c(c1)OCCO2]

However, in certain examples, context or express disclosure may indicate that the point of attachment is through a non-aromatic portion of the condensed ring system. For example,

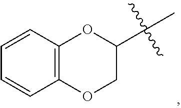 [SMILES: *C1COc2ccccc2O1]

such as with a heterocyclic-aromatic group. An aromatic group or moiety may comprise only carbon atoms in the ring, such as in an aryl group or moiety, or it may comprise one or more ring carbon atoms and one or more ring heteroatoms comprising a lone pair of electrons (e.g., S, O, N, P, or Si), such as in a heteroaryl group or moiety. Aromatic groups may be substituted with one or more groups other than hydrogen, such as aliphatic, heteroaliphatic, haloaliphatic, haloheteroaliphatic, aromatic, or an organic functional group.

Aryl: An aromatic carbocyclic group comprising at least five carbon atoms to 15 carbon atoms (C₅-C₁₅), such as five to ten carbon atoms (C₅-C₁₀), having a single ring or multiple condensed rings, which condensed rings can or may not be aromatic provided that the point of attachment to a remaining position of the compounds disclosed herein is through an atom of the aromatic carbocyclic group. Aryl groups may be substituted with one or more groups other than hydrogen, such as aliphatic, heteroaliphatic, haloaliphatic, haloheteroaliphatic, aromatic, or an organic functional group.

Azepanyl:

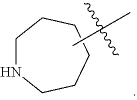

Autophagy: A process by which cells degrade and recycle cellular components, including damaged or dysfunctional organelles, proteins, and cell membrane. The targeted cellular components are isolated in autophagosomes, which fuse with lysosomes where the contents are degraded and recycled. Autophagy is believed to play a role in pathologies such as cancer and neurodegenerative disorders.

Breast cancer: A malignant neoplasm that arises in or from breast tissue (such as a ductal carcinoma). Breast cancers are frequently classified as luminal A (ER positive and/or PR positive, ErbB2 negative, and low Ki67), luminal B (ER positive and/or PR positive and ErbB2 positive, or ErbB2 negative with high Ki67), basal-like or triple-negative (ER negative, PR negative, ErbB2 negative, cytokeratin 5/6 positive and/or HER1 positive), or ErbB2 positive (ER negative, PR negative, ErbB2 positive). However, breast cancers may be heterogeneous both between individuals and at the cellular level within a tumor, and may not always fit within the classification scheme.

“Triple negative breast cancer” (TNBC) is a subtype of breast cancer characterized by lack of expression of estrogen and progesterone receptors (ER/PR) and lack expression of (or lack overexpression of) human epidermal growth factor receptor-2 (referred to as HER2 or ErbB2) in the tumor cells. In some examples, TNBC is invasive ductal carcinoma or ductal carcinoma in situ. In other examples, TNBC is basal-like breast cancer. The pathological features of TNBC may include lymphocytic infiltrate, pushing borders, high mitotic rate (>19/10 HPF), central necrosis, medullary features, and metaplastic elements (e.g., squamous cells and spindle cells).

Cancer: A malignant neoplasm that has undergone anaplasia with loss of differentiation, increased rate of growth, invasion of surrounding tissue, and is capable of metastasis. As used herein, cancer includes both solid tumors and hematological malignancies. Residual cancer is cancer that remains in a subject after any form of treatment is given to the subject to reduce or eradicate cancer. Metastatic cancer is a cancer at one or more sites in the body other than the original site of the cancer from which the metastatic cancer is derived. Local recurrence is a reoccurrence of the cancer at or near the same site as the original cancer, for example, in the same tissue as the original cancer.

Carbazolyl: H

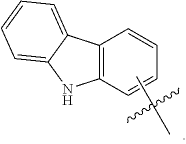

Diazabicyclooctanyl:

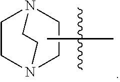

Dibenzofuranyl:

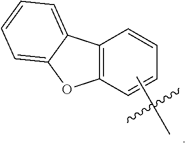

Dihydrobenzodioxinyl:

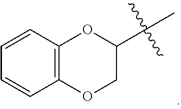 [SMILES: *C1COc2ccccc2O1]

Furanyl:

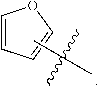

Glioblastoma: Also known as glioblastoma multiforme (GBM), which accounts for about 15% of brain tumors. GBM are aggressive tumors, with average survival of about 12-15 months following diagnosis. The pathological features of GBM include small areas of necrotizing tumor surrounded by anaplastic cells and the presence of hyperplastic blood vessels. GBM is difficult to treat, in part due to the limited ability of most drugs to cross the blood-brain barrier and contact the tumor.

Halo (or halide or halogen): Fluoro, chloro, bromo, or iodo.

Haloaliphatic: An aliphatic group wherein one or more hydrogen atoms, such as one to 10 hydrogen atoms, independently is replaced with a halogen atom, such as fluoro, bromo, chloro, or iodo.

Haloalkyl: An alkyl group wherein one or more hydrogen atoms, such as one to 10 hydrogen atoms, independently is replaced with a halogen atom, such as fluoro, bromo, chloro, or iodo. In an independent embodiment, haloalkyl can be a CX₃ group, wherein each X independently can be selected from fluoro, bromo, chloro, or iodo.

Haloalkenyl: An alkenyl group wherein one or more hydrogen atoms, such as one to 10 hydrogen atoms, independently is replaced with a halogen atom, such as fluoro, bromo, chloro, or iodo.

Haloalkynyl: An alkynyl group wherein one or more hydrogen atoms, such as one to 10 hydrogen atoms, independently is replaced with a halogen atom, such as fluoro, bromo, chloro, or iodo.

Heteroaliphatic: An aliphatic group comprising at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group. Alkoxy, ether, amino, disulfide, peroxy, and thioether groups are exemplary (but non-limiting) examples of heteroaliphatic.

Heteroalkenyl: An alkenyl group comprising at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group.

Heteroalkynyl: An alkynyl group comprising at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group.

Heteroaryl: An aryl group comprising at least one heteroatom to six heteroatoms, such as one to four heteroatoms, which can be selected from, but not limited to oxygen, nitrogen, sulfur, silicon, boron, selenium, phosphorous, and oxidized forms thereof within the ring. Such heteroaryl groups can have a single ring or multiple condensed rings, wherein the condensed rings may or may not be aromatic and/or contain a heteroatom, provided that the point of attachment is through an atom of the aromatic heteroaryl group. Heteroaryl groups may be substituted with one or more groups other than hydrogen, such as aliphatic, heteroaliphatic, haloaliphatic, haloheteroaliphatic, aromatic, or an organic functional group.

Heteroatom: An atom other than carbon or hydrogen, such as (but not limited to) oxygen, nitrogen, sulfur, silicon, boron, selenium, or phosphorous. In particular disclosed embodiments, such as when valency constraints do not permit, a heteroatom does not include a halogen atom.

Neurodegenerative disease: A disease associated with the progressive loss of structure or function of neurons, including death of neurons. Examples of such diseases include Alzheimer's disease, Parkinson's disease, Huntington's disease, amyotrophic lateral sclerosis (ALS), tauopathies (such as progressive supranuclear palsy, corticobasal degeneration and frontotemporal dementia), spinocerebellar ataxias, spinal and bulbar muscular dystrophy, hereditary spastic paraplegias, Lafora disease, Charcot-Marie-Tooth disease, and AIDS dementia. In some examples, neurodegenerative disease is associated with impaired autophagy.

Pharmaceutically acceptable carrier: Remington: The Science and Practice of Pharmacy, The University of the Sciences in Philadelphia, Editor, Lippincott, Williams, & Wilkins, Philadelphia, Pa., 21^{st} Edition (2005), describes compositions and formulations suitable for pharmaceutical delivery of one or more therapeutic compounds or molecules. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions can contain minor amounts of non-toxic auxiliary substances, such as wetting or emulsifying agents, preservatives, pH buffering agents, and the like, for example sodium acetate or sorbitan monolaurate.

Piperidinyl:

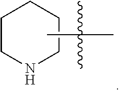

Pyridinyl:

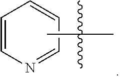

Pyrrole:

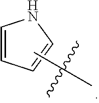

Quinolinyl:

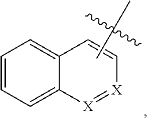

wherein each X independently is CH or N.

Subject: A living multi-cellular vertebrate organism, a category that includes both human and veterinary subjects, including human and non-human mammals.

Therapeutically effective amount or effective amount: A quantity of a specific substance, such as a therapeutic agent, sufficient to treat, reduce, and/or ameliorate the symptoms and/or underlying causes of a disorder or disease. In some embodiments, a therapeutically effective amount is the amount necessary to reduce or eliminate a symptom of a disease, such as cancer or a neurodegenerative disorder. In some examples, when administered to a subject, a dosage is used that will achieve target tissue concentration that has been shown to achieve a desired effect.

Thiophenyl:

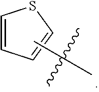

ULK1: unc-51 like autophagy activating kinase 1; also known as Unc51.1 or UNC51. ULK1 is a ubiquitously expressed serine/threonine protein kinase involved in autophagy. It is a 112-kDa protein that consists of an N-terminal kinase domain, a serine-proline rich region, and a C-terminal interacting domain (Zhang et al., J. Med. Chem. 61:6491-6500, 2018). ULK1 activity is negatively regulated by mammalian target of rapamycin complex 1 (mTORC1) and positively regulated by AMP-activated protein kinase (AMPK), depending on the phosphorylation sites in the serine-proline rich region (Kim et al., Nat. Cell Biol. 13:132-141, 2011). Stress signals are mediated through the C-terminal domain and autophagy is propagated through the ULK1-ATG13-FIP200 complex (Zhang et al., J. Med. Chem. 61:6491-65002018; He et al., J. Med. Chem. 61:4656-4687, 2017).

Xanthenyl:

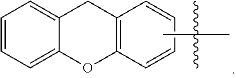

### II. Compositions

Disclosed herein are compositions that include VMY-BC-1 or derivatives thereof. In some embodiments, the compositions bind to and activate ULK1 and/or stimulate autophagy.

In some embodiments, the disclosure provides compounds for treating TNBC, glioblastoma, or neurodegenerative disease, such as a compound having the structure shown in Formula I (VMY-BC-1).

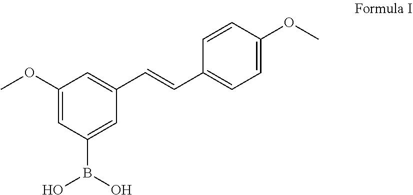

as well as all pharmaceutically acceptable salts, stereoisomers, prodrugs, and tautomers thereof.

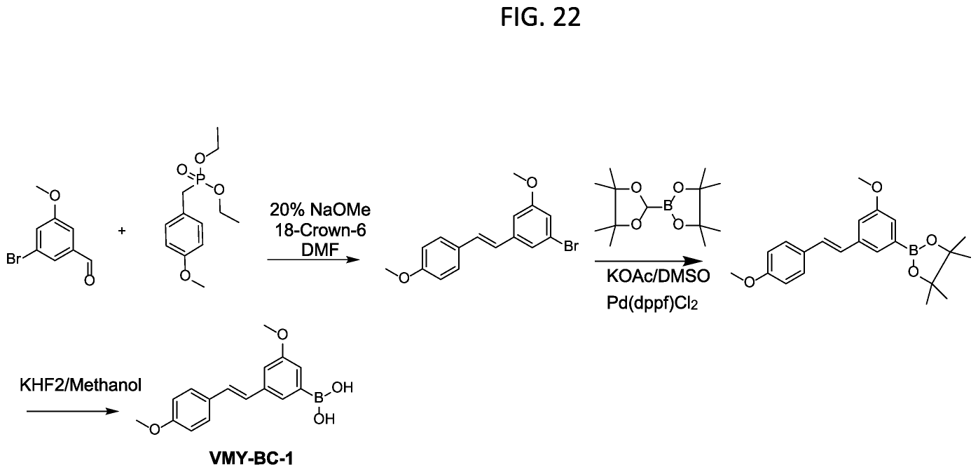

In some examples, VMY-BC-1 is synthesized as described in U.S. Pat. App. Publ. No. 2012/0149663. In other examples, VMY-BC-1 is synthesized by the synthetic scheme shown in FIG. 22.

In some embodiments, disclosed is a compound that can have a structure of Formula II, IIA, or IIB

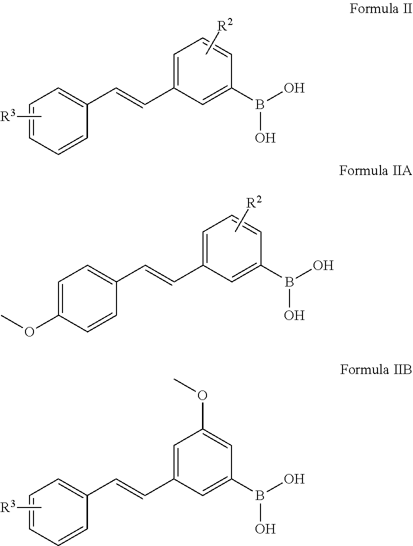

or a pharmaceutically acceptable salt, stereoisomer, tautomer, or prodrug thereof.

With reference to Formula II, IIA, and IIB, each of R² and R³ independently are selected from aliphatic, haloaliphatic, halogen, —C(O)OR^{b}, —C(O)N(R^{b})₂, —C(O)H, —OC(O)R^{b}, —OC(O)OR^{b}, —NR^{b}S(O)₂R^{b}, —S(O)₂N(R^{b})₂, —[(C(R^{b})₂)ₙO]ₘR^{b}, —OR^{b}, —SR^{b}, —N(R^{b})₂, wherein each R^{b} independently is hydrogen, aliphatic, heteroaliphatic, haloaliphatic, aromatic, or combinations thereof; and each of n and m independently is an integer ranging from 1 to 50, such as 1 to 25, or 1 to 20, or 1 to 15, or 1 to 10 (e.g., 1, 2, 3, 4, 5, 6, 7, 8, 9, or 10). In some embodiments, each R^{b} independently is hydrogen, alkyl, alkenyl, alkynyl, heteroalkyl, heteroalkenyl, heteroalkynyl, haloalkyl, haloalkenyl, haloalkynyl, aryl, heteroaryl, or combinations thereof.

In some embodiments, each of R² and R³ independently is selected from alkyl, —C(H)(halogen)₂, —C(H₂)(halogen), —C(halogen)₃, Cl, F, Br, I, —C(O)OH, —C(O)NH₂, —C(O)N(H)alkyl, —C(O)N(alkyl)₂, —C(O)O-alkyl, —OC(O)-alkyl, —OC(O)O-alkyl, —NHS(O)₂alkyl, —N(alkyl)S(O)₂alkyl, —S(O)₂NH₂, —S(O)₂N(alkyl)₂, —S(O)₂N(H)alkyl, [—(CH₂)ₙO-]ₘH, —OH, —O-alkyl, —O-heteroalkyl, —SH, —S-alkyl, —S-heteroalkyl, —NH₂, —N(alkyl)₂, or —N(H)alkyl, wherein each alkyl group independently is selected from lower alkyl, such as methyl, ethyl, propyl, butyl, pentyl, hexyl, and the like, including branched and/or cyclic versions thereof, and wherein the heteroalkyl group is —(CH₂)_{q}N(alkyl)₂, wherein q is an integer selected from 1, 2, or 3.

In some embodiments, each of R² and R³ independently is selected from methyl, ethyl, propyl, isopropyl, butyl, isobutyl, t-butyl, Cl, F, —CF₃, —COOH, —OC(O)Me, —C(O)NH₂, NH₂, —NMe₂, —NHMe, —SO₂NH₂, —OEt, —OPr, —OBu, —O(CH₂)₂N(Me)₂, or —OiPr.

Exemplary compounds are illustrated below.

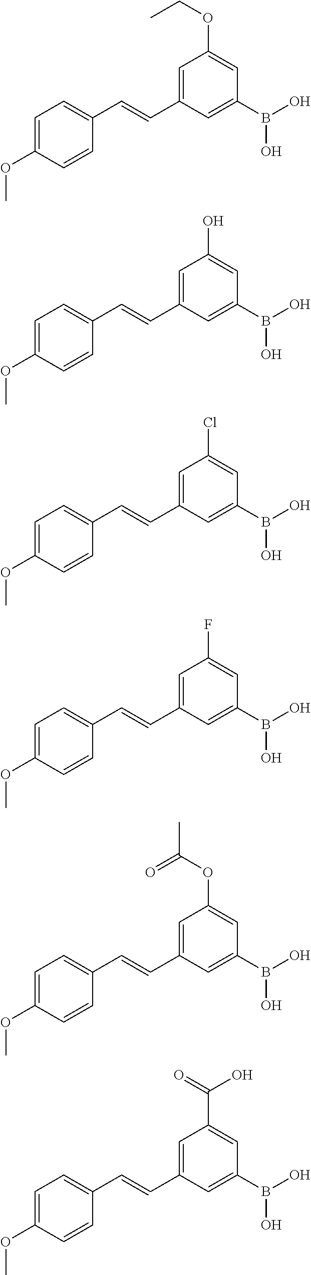 [SMILES: CCOc1cc(/C=C/c2ccc(OC)cc2)cc(B(O)O)c1 ; COc1ccc(/C=C/c2cc(O)cc(B(O)O)c2)cc1 ; COc1ccc(/C=C/c2cc(Cl)cc(B(O)O)c2)cc1 ; COc1ccc(/C=C/c2cc(F)cc(B(O)O)c2)cc1 ; COc1ccc(/C=C/c2cc(OC(C)=O)cc(B(O)O)c2)cc1 ; COc1ccc(/C=C/c2cc(B(O)O)cc(C(=O)O)c2)cc1]

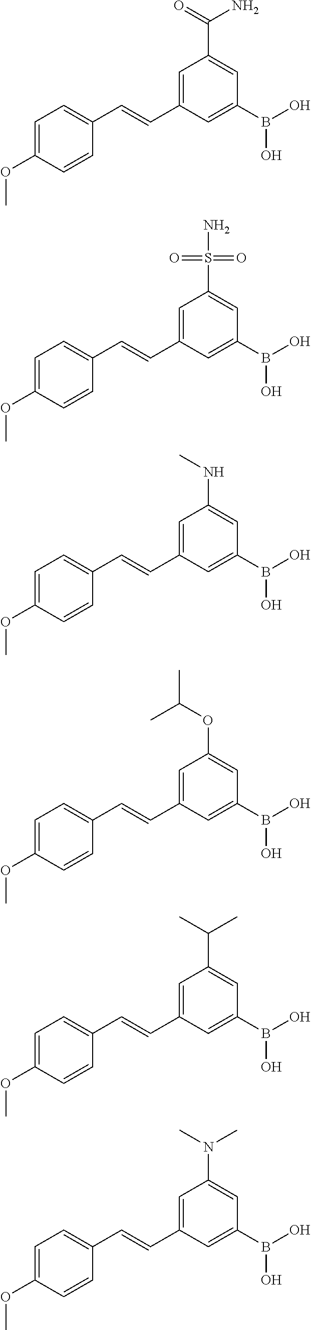 [SMILES: COc1ccc(/C=C/c2cc(B(O)O)cc(C(N)=O)c2)cc1 ; COc1ccc(/C=C/c2cc(B(O)O)cc(S(N)(=O)=O)c2)cc1 ; CNc1cc(/C=C/c2ccc(OC)cc2)cc(B(O)O)c1 ; COc1ccc(/C=C/c2cc(OC(C)C)cc(B(O)O)c2)cc1 ; COc1ccc(/C=C/c2cc(B(O)O)cc(C(C)C)c2)cc1 ; COc1ccc(/C=C/c2cc(B(O)O)cc(N(C)C)c2)cc1]

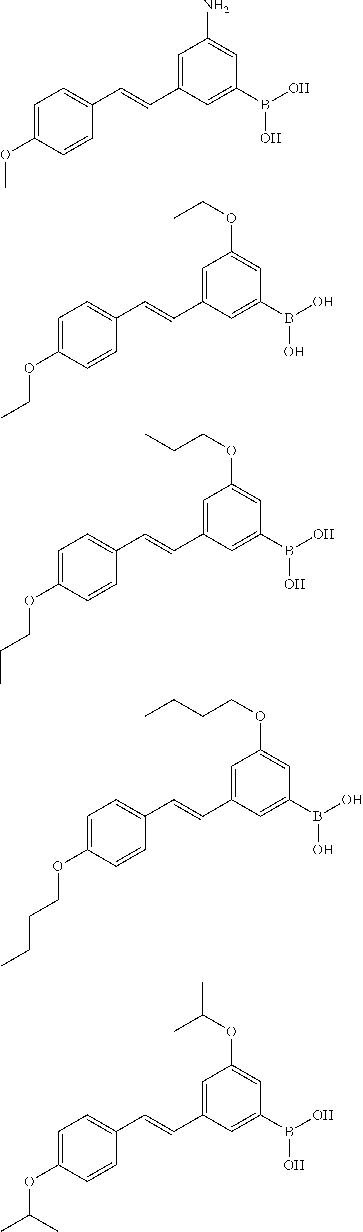 [SMILES: COc1ccc(/C=C/c2cc(N)cc(B(O)O)c2)cc1 ; CCOc1ccc(/C=C/c2cc(OCC)cc(B(O)O)c2)cc1 ; CCCOc1ccc(/C=C/c2cc(OCCC)cc(B(O)O)c2)cc1 ; CCCCOc1ccc(/C=C/c2cc(OCCCC)cc(B(O)O)c2)cc1 ; CC(C)Oc1ccc(/C=C/c2cc(OC(C)C)cc(B(O)O)c2)cc1]

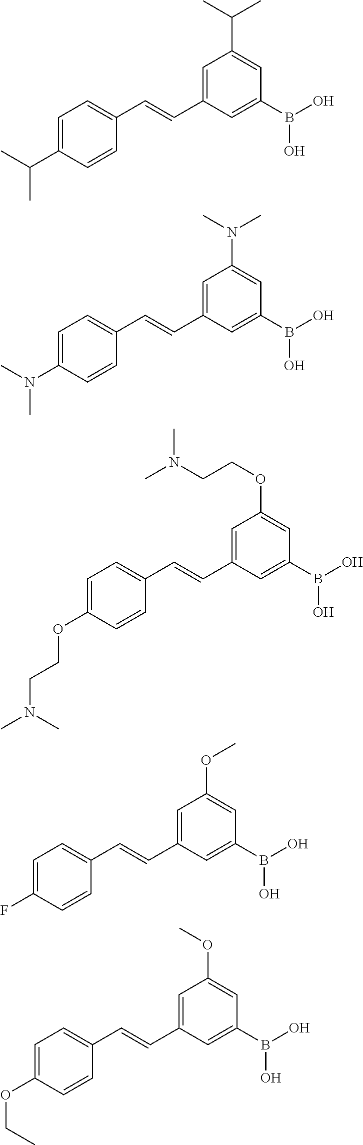 [SMILES: CC(C)c1ccc(/C=C/c2cc(B(O)O)cc(C(C)C)c2)cc1 ; CN(C)c1ccc(/C=C/c2cc(B(O)O)cc(N(C)C)c2)cc1 ; CN(C)CCOc1ccc(/C=C/c2cc(OCCN(C)C)cc(B(O)O)c2)cc1 ; COc1cc(/C=C/c2ccc(F)cc2)cc(B(O)O)c1 ; CCOc1ccc(/C=C/c2cc(OC)cc(B(O)O)c2)cc1]

In further embodiments, the compound has a structure of Formula III.

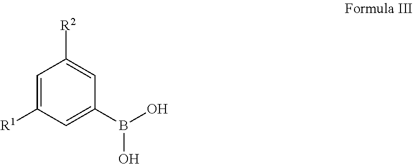

or a pharmaceutically acceptable salt, stereoisomer, tautomer, or prodrug thereof.

With reference to Formula III, R¹ is —[(CH═CH)-]ₚheterocyclic, —[(CH═CH)-]ₚaromatic, or —[(CH═CH)-]ₚheterocyclic-aromatic, wherein p is 0 or 1; and R² is as recited above for any of Formulas II, IIA, and/or IIB. In some embodiments, the R¹ heterocyclic group comprises 2 to 10 carbon atoms and one or more heteroatoms selected from oxygen, nitrogen, or combinations thereof. In some embodiments, the R¹ aromatic group is an aryl group or a heteroaryl group. In some embodiments, R¹ heterocyclic-aromatic group comprises one or more heterocyclic groups fused with one or more aromatic groups. In some embodiments, p is 0, R¹ is carbazolyl, dihydrobenzodioxinyl, dibenzofuranyl, or xanthenyl, and R² is methoxy.

In further embodiments, the compound has a structure of Formula IIIA.

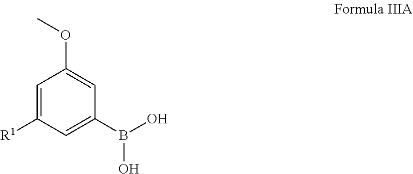

or a pharmaceutically acceptable salt, stereoisomer, tautomer, or prodrug thereof.

With reference to Formula IIIA, R¹ is —[(CH═CH)-]ₚheterocyclic, —[(CH═CH)-]ₚaromatic, or —[(CH═CH)-]ₚheterocyclic-aromatic, wherein p is 0 or 1. In some embodiments, the R¹ heterocyclic group comprises 2 to 10 carbon atoms and one or more heteroatoms selected from oxygen, nitrogen, or combinations thereof. In some embodiments, the R¹ aromatic group is an aryl group or a heteroaryl group. In some embodiments, R¹ heterocyclic-aromatic group comprises one or more heterocyclic groups fused with one or more aromatic groups. In some embodiments, p is 0 and R¹ is carbazolyl, dihydrobenzodioxinyl, dibenzofuranyl, or xanthenyl.

Exemplary compounds where p is 0 are illustrated below.

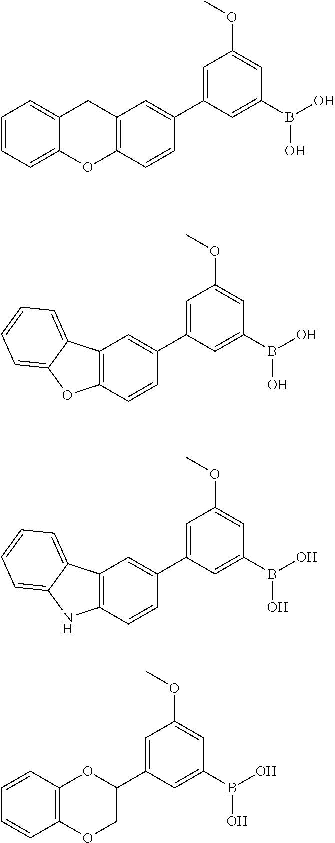 [SMILES: COc1cc(B(O)O)cc(-c2ccc3c(c2)Cc2ccccc2O3)c1 ; COc1cc(B(O)O)cc(-c2ccc3oc4ccccc4c3c2)c1 ; COc1cc(B(O)O)cc(-c2ccc3[nH]c4ccccc4c3c2)c1 ; COc1cc(B(O)O)cc(C2COc3ccccc3O2)c1]

In some embodiments, R¹ is —[(CH═CH)-]ₚheterocyclic, —[(CH═CH)-]ₚaromatic, or —[(CH═CH)-]ₚheterocyclic-aromatic, wherein p is 1. In such embodiments, the compound can have a structure of Formula IIIB or IIIC, wherein R⁴ is naphthyl, pyridinyl, pyrrole, furanyl, thiophenyl, quinolinyl, piperidinyl, azepanyl, or diazabicyclooctanyl and R² is as recited above for Formulas II, IIA, and/or IIB.

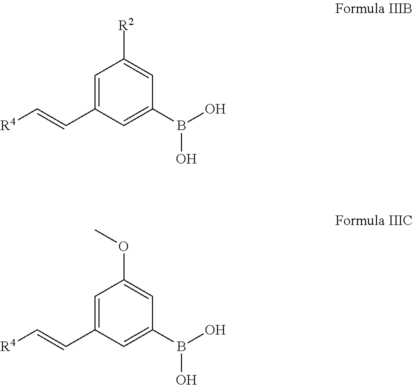

Exemplary compounds of Formula IIIA and Formula IIIB are illustrated below.

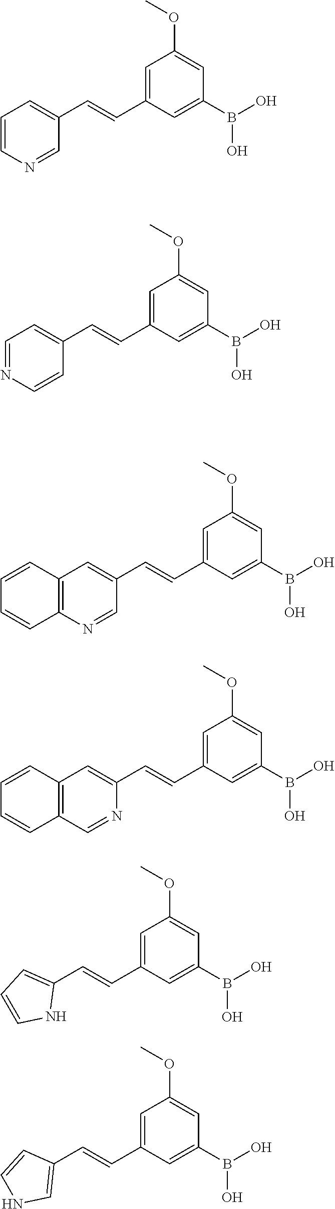 [SMILES: COc1cc(/C=C/c2cccnc2)cc(B(O)O)c1 ; COc1cc(/C=C/c2ccncc2)cc(B(O)O)c1 ; COc1cc(/C=C/c2cnc3ccccc3c2)cc(B(O)O)c1 ; COc1cc(/C=C/c2cc3ccccc3cn2)cc(B(O)O)c1 ; COc1cc(/C=C/c2ccc[nH]2)cc(B(O)O)c1 ; COc1cc(/C=C/c2cc[nH]c2)cc(B(O)O)c1]

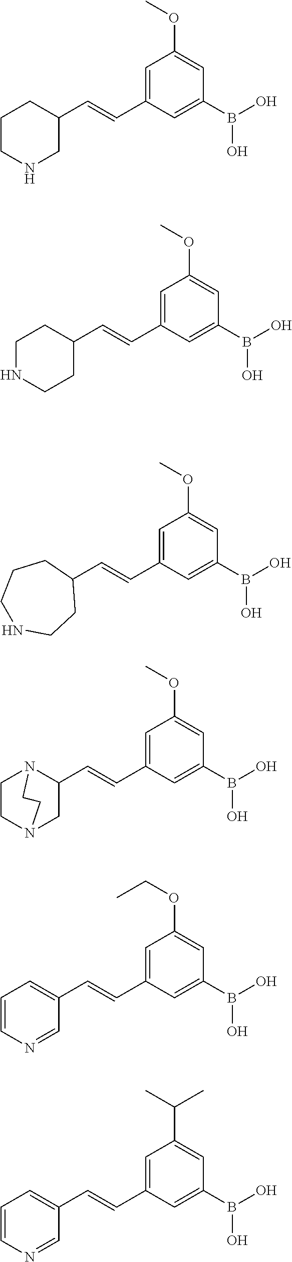 [SMILES: COc1cc(/C=C/C2CCCNC2)cc(B(O)O)c1 ; COc1cc(/C=C/C2CCNCC2)cc(B(O)O)c1 ; COc1cc(/C=C/C2CCCNCC2)cc(B(O)O)c1 ; COc1cc(/C=C/C2CN3CCN2CC3)cc(B(O)O)c1 ; CCOc1cc(/C=C/c2cccnc2)cc(B(O)O)c1 ; CC(C)c1cc(/C=C/c2cccnc2)cc(B(O)O)c1]

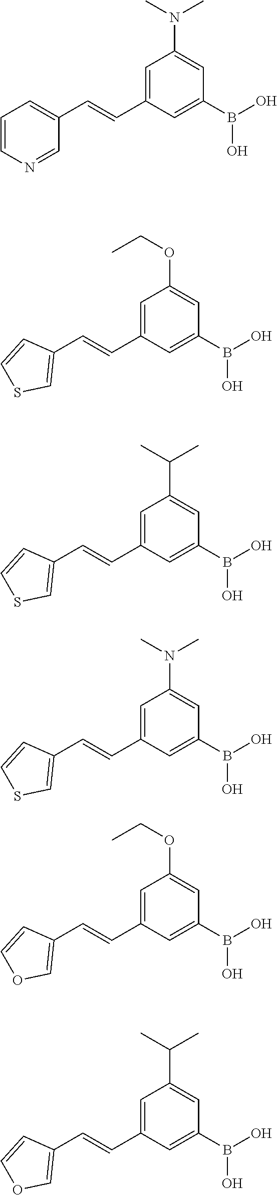 [SMILES: CN(C)c1cc(/C=C/c2cccnc2)cc(B(O)O)c1 ; CCOc1cc(/C=C/c2ccsc2)cc(B(O)O)c1 ; CC(C)c1cc(/C=C/c2ccsc2)cc(B(O)O)c1 ; CN(C)c1cc(/C=C/c2ccsc2)cc(B(O)O)c1 ; CCOc1cc(/C=C/c2ccoc2)cc(B(O)O)c1 ; CC(C)c1cc(/C=C/c2ccoc2)cc(B(O)O)c1]

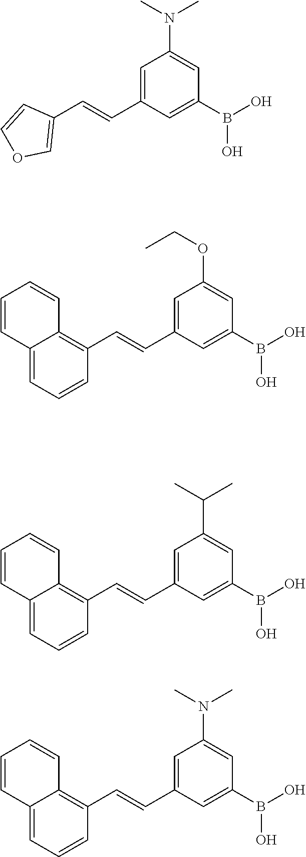 [SMILES: CN(C)c1cc(/C=C/c2ccoc2)cc(B(O)O)c1 ; CCOc1cc(/C=C/c2cccc3ccccc23)cc(B(O)O)c1 ; CC(C)c1cc(/C=C/c2cccc3ccccc23)cc(B(O)O)c1 ; CN(C)c1cc(/C=C/c2cccc3ccccc23)cc(B(O)O)c1]

In additional embodiments, the compound has a structure of Formula IV

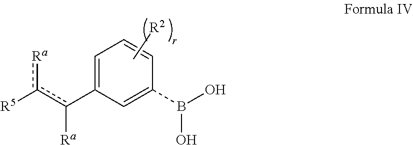

or a pharmaceutically acceptable salt, stereoisomer, tautomer, or prodrug thereof.

With reference to Formula IV, each R^{a} independently is hydrogen, halogen, aliphatic, heteroaliphatic, haloaliphatic, aromatic, or any combination thereof; R² is as described above for any of Formulas II, IIA, and/or IIB; R⁵ is either an R⁴ group as described herein for Formulas IIIB and/or IIIC, or is an aromatic group comprising an R³ substituent, wherein the R³ group is as recited herein for Formulas II, IIA, and/or IIB; and r is an integer selected from 0 or 1. In some embodiments, each R^{a} independently is halogen, aliphatic, aromatic, or any combination thereof. In particular disclosed embodiments, each R^{a} independently is hydrogen, F, Br, Cl, I, alkyl (e.g., lower alkyl, such as methyl, ethyl, propyl, butyl, pentyl, hexyl, and the like, including branched and/or cyclic versions thereof), heteroalkyl, aryl (e.g., phenyl), or combinations thereof. In particular disclosed embodiments, r is 1 and R² is isopropyl, —NMe₂, —OEt, —OPr, —OBu, —O(CH₂)₂N(Me)₂, or —OiPr. In particular disclosed embodiments, R⁵ is -Ph-(R³)ᵣ, wherein R³ is in the para position and is selected from isopropyl, —NMe₂, —OEt, —OPr, —OBu, —O(CH₂)₂N(Me)₂, or —OiPr. In yet other disclosed embodiments, R⁵ is naphthyl, pyridinyl, furanyl, or thiophenyl. In some embodiments, each R^{a} independently is F, ethyl, phenyl, or -PhO(CH₂)₂NMe₂. In some embodiments, each R^{a} is different.

In some embodiments, the compound of Formula IV can further have a structure of Formula IVA,

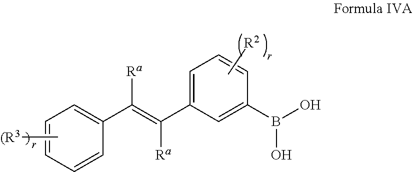

or a pharmaceutically acceptable salt, stereoisomer, tautomer, or prodrug thereof.

With reference to Formula IVA, each R^{a} independently is hydrogen, halogen, aliphatic, heteroaliphatic, haloaliphatic, aromatic, or any combination thereof; each of R² and R³ independently is as described above for any of Formulas II, IIA, and/or IIB; and each r independently is an integer selected from 0 or 1. In some embodiments, each R^{a} independently is halogen, aliphatic, aromatic, or any combination thereof. In particular disclosed embodiments, each R^{a} independently is hydrogen, F, Br, Cl, I, alkyl (e.g., lower alkyl, such as methyl, ethyl, propyl, butyl, pentyl, hexyl, and the like, including branched and/or cyclic versions thereof), heteroalkyl, aryl (e.g., phenyl), or combinations thereof. In particular embodiments, each r is 1 and each of R² and R³ independently is isopropyl, —NMe₂, —OEt, —OPr, —OBu, —O(CH₂)₂N(Me)₂, or —OiPr. In some embodiments, each R^{a} independently is F, ethyl, phenyl, or -PhO(CH₂)₂NMe₂. In some embodiments, each R^{a} is different.

In some embodiments, a compound having a structure of Formula IV can further have a structure of Formula IVB, IVC IVD IVE or IVF

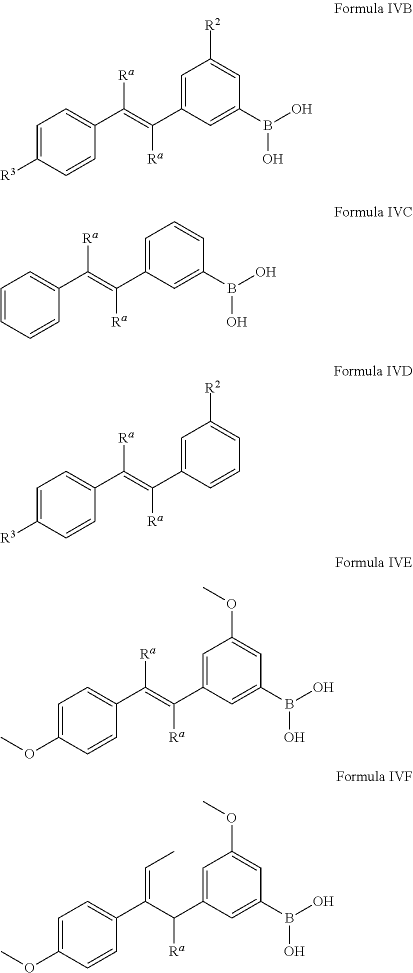

or a pharmaceutically acceptable salt, stereoisomer, tautomer, or prodrug thereof.

With reference to Formulas IVB, IVC, IVD, and/or IVE, each R² of R³ independently is as recited for any of the formulas described above; each R^{a} independently is hydrogen, halogen, aliphatic, heteroaliphatic, haloaliphatic, aromatic, or any combination thereof. In some embodiments, each R^{a} independently is halogen, aliphatic, aromatic, or any combination thereof. In particular disclosed embodiments, each R^{a} independently is hydrogen, F, Br, Cl, I, alkyl (e.g., lower alkyl, such as methyl, ethyl, propyl, butyl, pentyl, hexyl, and the like, including branched and/or cyclic versions thereof), heteroalkyl, aryl (e.g., phenyl), or combinations thereof. In some embodiments, each R^{a} independently is F, ethyl, phenyl, or -PhO(CH₂)₂NMe₂. In some embodiments, each R^{a} is different. In some embodiments of Formula IVB or IVD, R² and R³ are the same and are selected from isopropyl, —NMe₂, —OEt, —OPr, —OBu, —O(CH₂)₂N(Me)₂, or —OiPr.

Exemplary compounds are illustrated below.

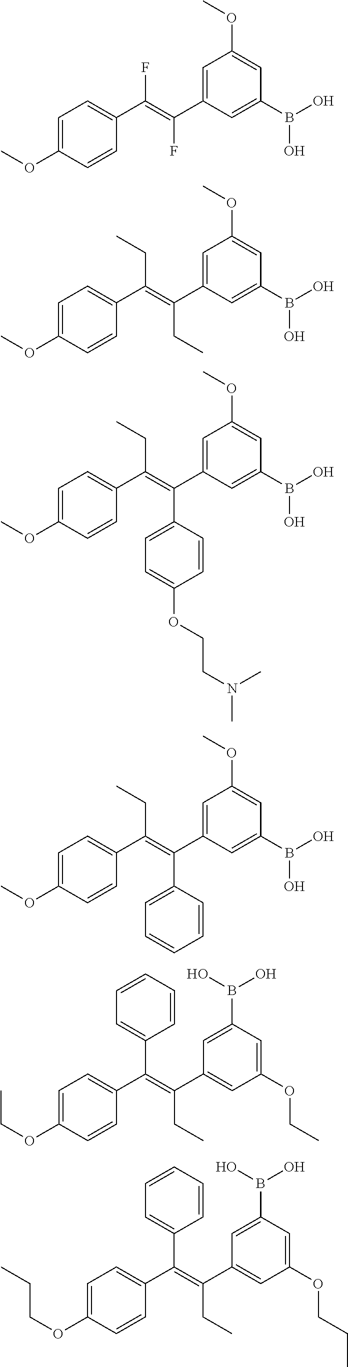

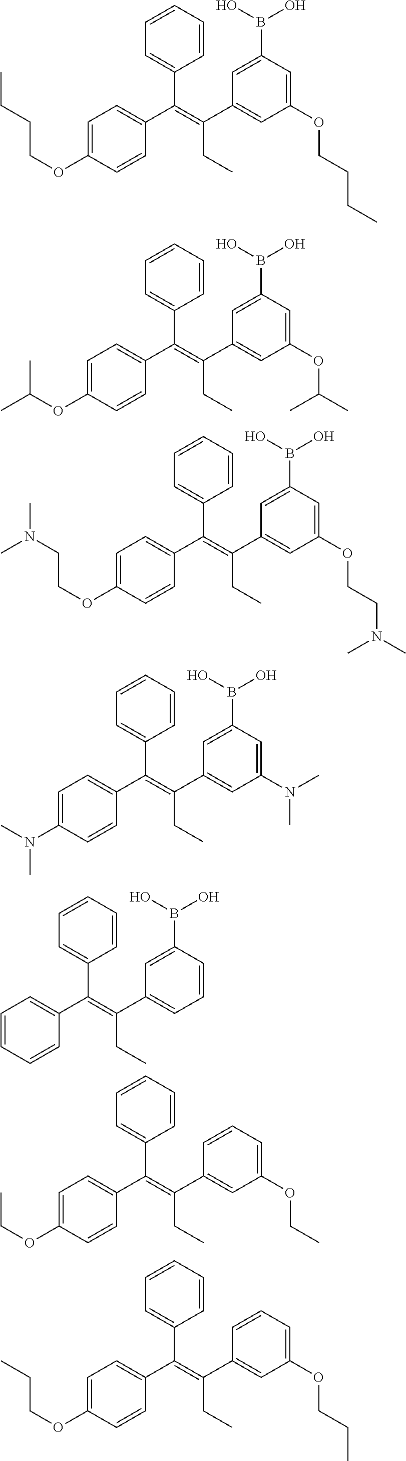

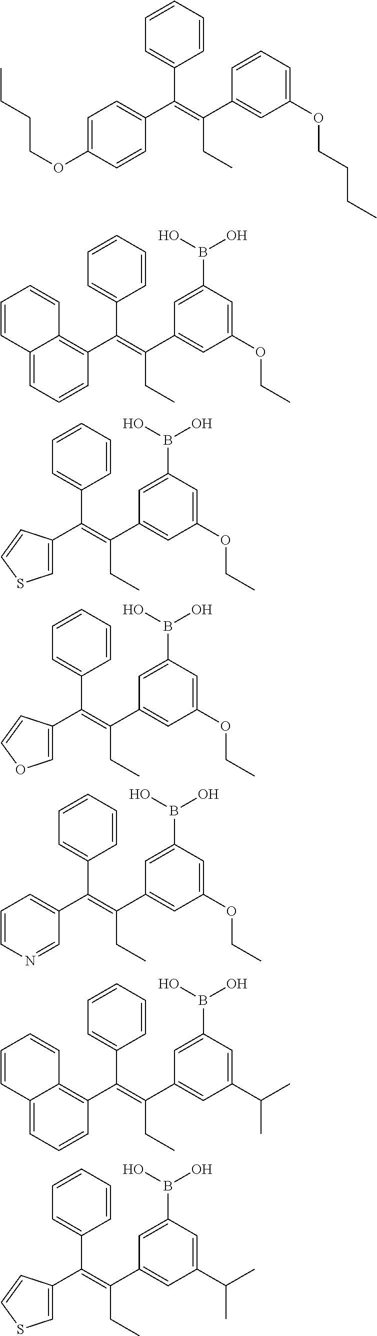

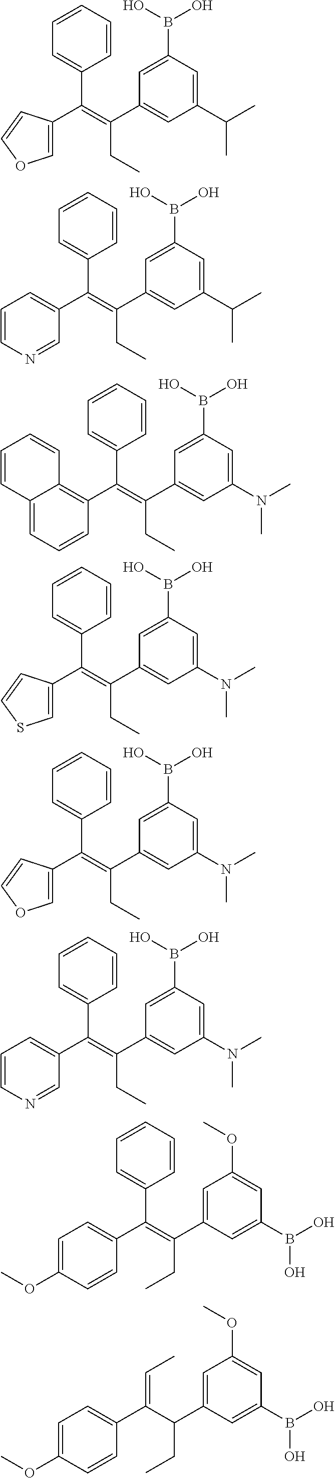

“Pharmaceutically acceptable salts” of the presently disclosed compounds include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Description of suitable pharmaceutically acceptable salts can be found in Handbook of Pharmaceutical Salts, Properties, Selection and Use, Wiley VCH (2002).

“Prodrug” refers to compounds that are transformed in vivo to yield a biologically active compound, particularly the parent compound, for example, by hydrolysis in the gut or enzymatic conversion. Any chemical compound recited in this specification may alternatively be administered as a prodrug thereof. Common examples of prodrug moieties include, but are not limited to, ester and amide forms of a compound having an active form bearing a carboxylic acid moiety. Examples of pharmaceutically acceptable esters of the disclosed compounds include, but are not limited to, esters of phosphate groups and carboxylic acids, such as aliphatic esters, particularly alkyl esters (for example C₁₋₆alkyl esters). Other prodrug moieties include phosphate esters, such as —CH₂—O—P(O)(OR′)₂ or a salt thereof, wherein R′ is H or C₁-6alkyl. Acceptable esters also include cycloalkyl esters and arylalkyl esters such as, but not limited to benzyl. Examples of pharmaceutically acceptable amides of the disclosed compounds include, but are not limited to, primary amides, and secondary and tertiary alkyl amides (for example with between about one and about six carbons). Amides and esters of disclosed exemplary embodiments of compounds can be prepared. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

In some non-limiting examples, the disclosed compounds activate ULK1 and/or increase autophagy. For example, as disclosed in the Examples (below), VMY-BC-1 is a compound that increases ULK1 phosphorylation and autophagy. Methods of determining whether a compound activates ULK1 and/or increases autophagy include those described in the Examples. In one example, activation of ULK1 is determined by detecting an increase in phosphorylation of ULK1 (for example, by Western blot) compared to a resting state or a control. For example, activation of ULK1 by a compound can be determined by treating a cell expressing ULK1 with a compound of interest and detecting phosphorylation of ULK1 compared to an untreated cell or a cell treated with a control compound (such as vehicle). In other examples, an increase in autophagy is determined by detecting an increase in phosphorylation of ULK1, increased expression of LC3, and/or increase in cleaved PARP (for example by Western blotting). For example, an increase in autophagy can be determined by treating a cell with a compound of interest and detecting phosphorylation of ULK1, increased expression of LC3, and/or increased cleaved PARP compared to an untreated cell or a cell treated with a control compound (such as vehicle). In other examples, an increase in autophagy can be determined by treating a cell with a compound of interest and detecting expression and/or phosphorylation of p62, beclin-1, autophagy-related protein (ATG, e.g., ATG13 or ATG101), AMPK, and/or mTOR compared to an untreated cell or a cell treated with a control compound (such as vehicle). See, e.g., Zachari et al., Essays in Biochemistry 61:585-596, 2017.

### III. Methods of Treatment

Disclosed herein are methods of treating cancer or neurodegenerative disease. In some embodiments, the methods include administering an effective amount of a composition that increases autophagy to a subject with cancer or a neurodegenerative disease.

Disclosed are methods of treating triple-negative breast cancer, brain cancer (for example, glioblastoma multiforme) with compositions including VMY-BC-1 (Formula I). Also disclosed are methods of treating cancer with compositions including derivatives or analogs of VMY-BC-1. In some embodiments, the methods include administering an effective amount of a composition including the compound of Formula I to a subject with TNBC or brain cancer (such as GBM). In further embodiments, the methods include administering an effective amount of a composition including the compound of any one of Formulas II-IV to a subject with cancer.

In some examples, the subject being treated has cancer, such as a solid tumor or a hematological malignancy. Examples of solid tumors, include sarcomas (such as fibrosarcoma, myxosarcoma, liposarcoma, chondrosarcoma, osteosarcoma, soft tissue sarcoma, and other sarcomas), synovioma, mesothelioma, Ewing sarcoma, leiomyosarcoma, rhabdomyosarcoma, colon cancer, colorectal cancer, peritoneal cancer, esophageal cancer (such as esophageal squamous cell carcinoma), pancreatic cancer, breast cancer (including basal breast carcinoma, ductal carcinoma and lobular breast carcinoma), endometrial cancer, lung cancer (such as non-small cell lung cancer), ovarian cancer, prostate cancer, liver cancer (including hepatocellular carcinoma), gastric cancer, squamous cell carcinoma (including head and neck squamous cell carcinoma), basal cell carcinoma, adenocarcinoma, sweat gland carcinoma, medullary thyroid carcinoma, papillary thyroid carcinoma, pheochromocytoma, sebaceous gland carcinoma, papillary carcinoma, papillary adenocarcinoma, medullary carcinoma, bronchogenic carcinoma, hepatoma, bile duct carcinoma, choriocarcinoma, Wilms tumor, cervical cancer, fallopian tube cancer, testicular tumor, seminoma, bladder cancer (such as renal cell cancer), melanoma, and CNS tumors (such as a glioma, glioblastoma, astrocytoma, medulloblastoma, craniopharyrgioma, ependymoma, pinealoma, hemangioblastoma, acoustic neuroma, oligodendroglioma, meningioma, neuroblastoma and retinoblastoma). Solid tumors also include tumor metastases (for example, metastases to the lung, liver, brain, or bone). In some examples, the subject has hepatocellular carcinoma, neuroblastoma, breast cancer, gastric cancer, endometrial cancer, bladder cancer (such as renal cell carcinoma), lung cancer (such as non-small cell lung cancer), cervical cancer, medulloblastoma, esophageal cancer (such as esophageal squamous cell carcinoma), prostate cancer, seminoma, glioblastoma, osteosarcoma, astrocytoma, or soft tissue sarcoma. In some particular examples, the subject has triple-negative breast cancer or brain cancer, such as glioblastoma.

Examples of hematological malignancies include leukemias, including acute leukemias (such as 11q23-positive acute leukemia, acute lymphocytic leukemia (ALL), T-cell ALL, acute myelocytic leukemia, acute myelogenous leukemia (AML), and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), lymphoblastic leukemia, polycythemia vera, lymphoma, diffuse large B cell lymphoma, Burkitt lymphoma, T cell lymphoma, follicular lymphoma, mantle cell lymphoma, Hodgkin disease, non-Hodgkin lymphoma, multiple myeloma, Waldenstrom macroglobulinemia, heavy chain disease, myelodysplastic syndrome, hairy cell leukemia, and myelodysplasia.

In some examples, the subject with cancer is treated with one or more additional therapies, such as surgery, radiation therapy, chemotherapy, and/or immunotherapy. A clinician can select appropriate treatments for the subject based on the type of cancer, the stage of cancer, response to prior therapies, the condition of the patient, and other factors. In one specific non-limiting example, the subject has TNBC and is treated with a compound of any one of Formulas I-IV and is also treated with surgery and/or chemotherapy (such as cisplatin or carboplatin).

Also disclosed are methods of treating a neurodegenerative disease. In some embodiments, an effective amount of a composition including the compound of any one of Formulas I-IV is administered to a subject with a neurodegenerative disease. Exemplary neurodegenerative diseases include Alzheimer's disease, Parkinson's disease, Huntington's disease, amyotrophic lateral sclerosis (ALS), tauopathies (such as progressive supranuclear palsy, corticobasal degeneration and frontotemporal dementia), spinocerebellar ataxias, spinal and bulbar muscular dystrophy, hereditary spastic paraplegias, Lafora disease, Charcot-Marie-Tooth disease, and AIDS dementia. In particular examples, the neurodegenerative disease is associated with presence of protein aggregation in the brain of the subject and treatment with one or more of the disclosed compositions decreases the presence or amount of protein aggregations in the brain.

The disclosed compounds can be formulated as pharmaceutical compositions for administration to a subject. In some examples, one or more of the provided compounds are combined with a pharmaceutically acceptable carrier or vehicle for administration to human or animal subjects. Examples of suitable pharmaceutically acceptable carriers, vehicles, or excipients include sterile aqueous or non-aqueous solutions, suspensions, and/or emulsions. Examples of non-aqueous solvents include propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present, such as surfactants, wetting or emulsifying agents (e.g., sorbitan monolaurate), buffers (e.g., sodium acetate), antimicrobials, anti-oxidants, chelating agents, inert gases, and the like. Remington: The Science and Practice of Pharmacy, The University of the Sciences in Philadelphia, Editor, Lippincott, Williams, & Wilkins, Philadelphia, Pa., 21^{st} Edition (2005), describes compositions and formulations suitable for pharmaceutical delivery of one or more therapeutic compounds or molecules.

Pharmaceutical compositions for oral use can also be formulated, for example, as tablets, troches, lozenges, aqueous or oily suspensions, dispersible powders or granules, emulsion hard or soft capsules, or syrups or elixirs. Such compositions may contain one or more agents selected from the group of sweetening agents, flavoring agents, coloring agents and preserving agents in order to provide pharmaceutically elegant and palatable preparations. Tablets contain the active ingredient in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatin or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monostearate or glyceryl distearate may be employed. Pharmaceutical compositions for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium such as peanut oil, liquid paraffin or olive oil.

The disclosed compounds can be administered by any means, such as by intravenous, intramuscular, subcutaneous, or intraperitoneal injection, or by oral, nasal, or anal administration. The disclosed compounds can also be administered topically, transdermally, or by local injection. In some embodiments, administration is oral (including buccal and/or sublingual administration), rectal, parenteral, aerosol, nasal, intravenous, intramuscular, subcutaneous, intradermal, or topical routes. To extend the time during which the compound is available to inhibit or treat a condition, the compound can be provided as an implant, an oily injection, a liposome, or as a particulate system. The particulate system can be a microparticle, a microcapsule, a microsphere, a nanoparticle, a nanocapsule, or similar particle.

In another embodiment, it may be desirable to administer the compounds or pharmaceutical compositions locally to the area in need of treatment. This may be achieved by, for example, local or regional infusion or perfusion, topical application, injection, catheter, suppository, or implant (e.g., implants formed from porous, non-porous, or gelatinous materials, including membranes, such as sialastic membranes or fibers), and the like.

In yet another embodiment, the compounds or pharmaceutical compositions can be delivered in a controlled release system. In one embodiment, a pump can be used (see, e.g., Langer Science 249:1527-1533, 1990; Sefton Crit. Rev. Biomed. Eng. 14:201-240, 1987; Buchwald et al., Surgery 88:507-516, 1980; Saudek et al., N. Engl. J. Med. 321:574-579, 1989). In another embodiment, polymeric materials can be used (see, e.g., Ranger et al., Macromol. Sci. Rev. Macromol. Chem. 23:61-64, 1983; Levy et al., Science 228:190-192, 1985; During et al., Ann. Neurol. 25:351-356, 1989; and Howard et al., J. Neurosurg. 71:105-112, 1989). Other controlled release systems, such as those discussed in the review by Langer (Science 249:1527-1533, 1990), can also be used.

Appropriate dosages for the disclosed compositions can be determined. The specific dose level and frequency of dosage for any particular subject may be varied and will depend upon a variety of factors, including the activity of the specific compound, the metabolic stability and length of action of that compound, the particular disease or disorder to be treated, the general health of the subject, the mode and time of administration, rate of excretion, and/or any drug combinations administered. Treatment can involve daily or multi-daily, weekly, bi-monthly, or monthly doses of compound(s) over a period of a few days or weeks to months, or even years.

The disclosed compounds can be conveniently presented in unit dosage form and prepared using conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers. The formulations may be included in unit-dose or multi-dose containers, for example, sealed ampoules and vials, and may be stored in a dried condition requiring only the addition of a sterile liquid carrier, for example, water or saline for injections, immediately prior to use. In certain embodiments, unit dosage formulations are those containing a dose or unit, or an appropriate fraction thereof, of the administered ingredient.

The amount of the compound that will be effective depends on the nature of the disorder or condition to be treated, as well as the stage of the disorder or condition. Effective amounts can be determined by in vitro studies, animal studies, and clinical techniques. The precise dose of the compounds to be included in the formulation will also depend on the route of administration, and should be decided according to the judgment of the health care practitioner and each subject's circumstances. An example of such a dosage range is 1 μg/kg to 200 mg/kg body weight (for example, about 5 μg/kg to 1 mg/kg, about 10 μg/kg to 5 mg/kg, about 100 μg/kg to 20 mg/kg, about 0.2 to 100 mg/kg, about 0.5 to 50 mg/kg, about 1 to 25 mg/kg, about 5 to 75 mg/kg, about 50 to 150 mg/kg, or about 100 to 200 mg/kg) in single or divided doses. For example, a suitable dose may be about 0.1 mg/kg, about 0.2 mg/kg, about 0.5 mg/kg, about 0.75 mg/kg, about 1 mg/kg, about 2 mg/kg, about 5 mg/kg, about 7.5 mg/kg, about 10 mg/kg, about 15 mg/kg, about 25 mg/kg, about 50 mg/kg, about 75 mg/kg, about 100 mg/kg, about 150 mg/kg, or about 200 mg/kg.

In other examples, the dosage range is 50-500 mg/m² (for example, about 50 to 100 mg/m², about 75 to 150 mg/m², about 125 to 200 mg/m², about 175 to 250 mg/m², about 225 to 300 mg/m², about 275 to 350 mg/m², about 325 to 400 mg/m², about 375 to 450 mg/m², or about 425 to 500 mg/m²) in single or divided doses. For example, a suitable dose may be about 50 mg/m², about 75 mg/m², about 100 mg/m², about 125 mg/m², about 150 mg/m², about 175 mg/m², about 200 mg/m², about 225 mg/m², about 250 mg/m², about 275 mg/m², about 300 mg/m², about 325 mg/m², about 350 mg/m², about 375 mg/m², about 400 mg/m², about 425 mg/m², about 450 mg/m², about 475 mg/m², or about 500 mg/m². In one non-limiting example, the dose is about 200 to 300 mg/m². However, other higher or lower dosages also could be used, as can be determined by in vitro and/or in vivo testing. See also, Nair et al., J. Basic Clin. Pharm. 7:27-31, 2016.

### EXAMPLES

The following examples are provided to illustrate certain features and/or embodiments. These examples should not be construed to limit the disclosure to the particular features or embodiments described.

### Example 1

### Molecular Properties of VMY-BC-1

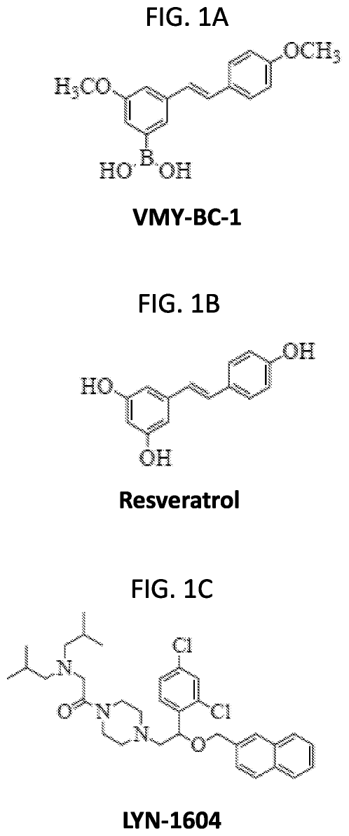

Molecular properties of VMY-BC-1 were compared with resveratrol and LYN-1604, a known small molecule ULK1 agonist (FIGS. 1A-1C and Tables 1 and 2; computed with SwissADME, Swiss Institute of Bioinformatics).

TABLE 1
Comparison of properties of VMY-BC-1 and LYN-1604

| Property | VMY-BC-1 | LYN-1604 |
|---|---|---|
| MW (g/mol) | 284.11 | 584.62 |
| Rotatable bonds | 5 | 13 |
| H-bond Acceptor | 4 | 4 |
| H-bond Donor | 2 | 0 |
| PTSA (A²) | 58.92 | 36.02 |
| Log P (ave) | 1.51 | 6.28 |
| Log S | −(3.9) | −7.72 |
| Water solubility (mg/ml) | 4.08e−02 | 5.03 e−08 |
| GI Absorption | High | High |
| BBB permeant | Yes | Yes |
| P-gp substrate | No | Yes |
| Lipinski (Pfizer) | Yes (no violations) | No: 2 violations |
| Veber (GS K) | Yes (no violations) | No: 1 violation |
| Egan (Pharmacia) | Yes (no violations) | No: 1 violation |
| Ghose | Yes (no violations) | No: 4 violations |
| Lead likeness | yes | No: 3 violations |

TABLE 2
Comparison of Central Nervous System
(CNS) and VMY-BC-1

| Property | CNS | VMY-BC-1 |
|---|---|---|
| MW (Da) | <450 | 284.11 |
| Log P | 1-3 | 1.51 |
| pKa | 7.5-10.5 | — |
| TPSA (A2) | <90 | 58 |
| Sum of N + O atoms | <5 | 4 |
| H-bond Acceptors | <6 | 4 |
| H-bond Donors | <2 | 2 |
| MPO desirability | 4-6 | 6 |

### Example 2

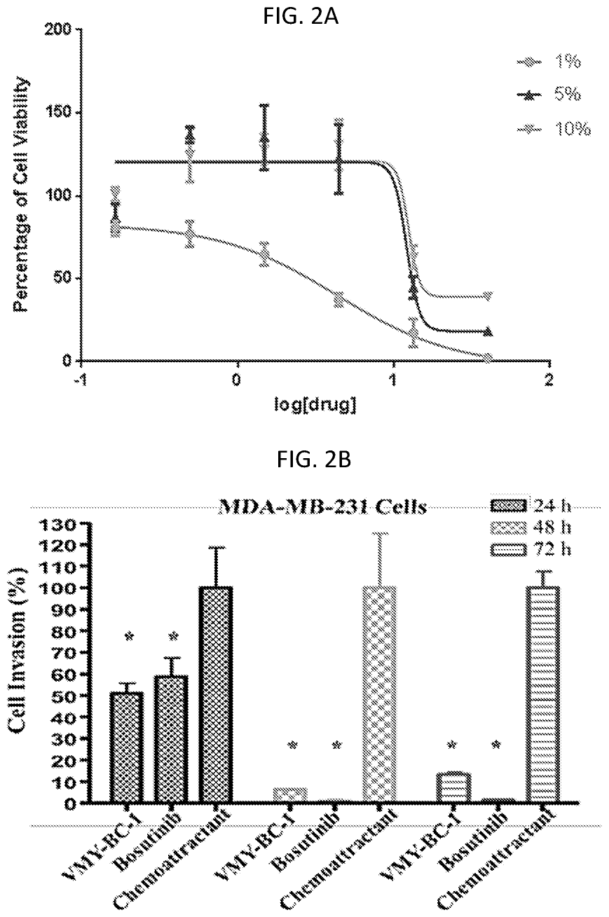

Effect of VMY-BC-1 on Triple Negative Breast Cancer Cell Lines A trans-stilbene boronic acid analog, VMY-BC-1, was tested against a TNBC cell line. VMY-BC-1 significantly inhibited growth of MDA-MB-231 TNBC cells (FIG. 2A). VMY-1BC-1 also significantly inhibited TNBC cell invasion in a time-dependent manner (FIG. 2B).

### Example 3

### Identifying Molecular Target of VMY-BC-1

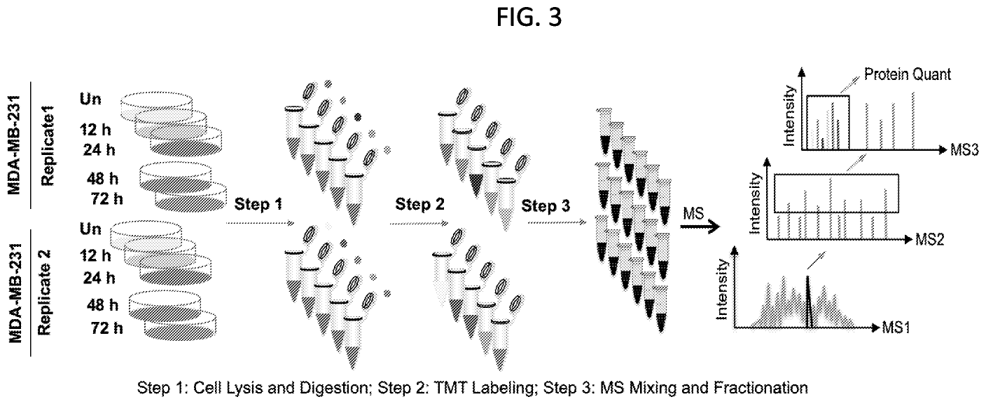

A global quantitative proteomics approach using multiplexed tandem mass tagged (TMT) isotope labeling technology and shotgun mass spectrometry (Hung et al., Anal. Chem. 84:161-170, 2012) (FIG. 3) was used to quantitate approximately 5,400 proteins. MDA-MB-231 cells were treated with vehicle control (DMSO) or 10 μM VMY-BC-1 for 4 time points. Equal amounts of protein from cell lysates were trypsin digested (Step 1) and labelled with TMT 10-plex reagent (Step 2). Labelled peptide samples were analyzed by shotgun mass spectrometry (Step 3) which resulted in quantification of ˜5,400 proteins.

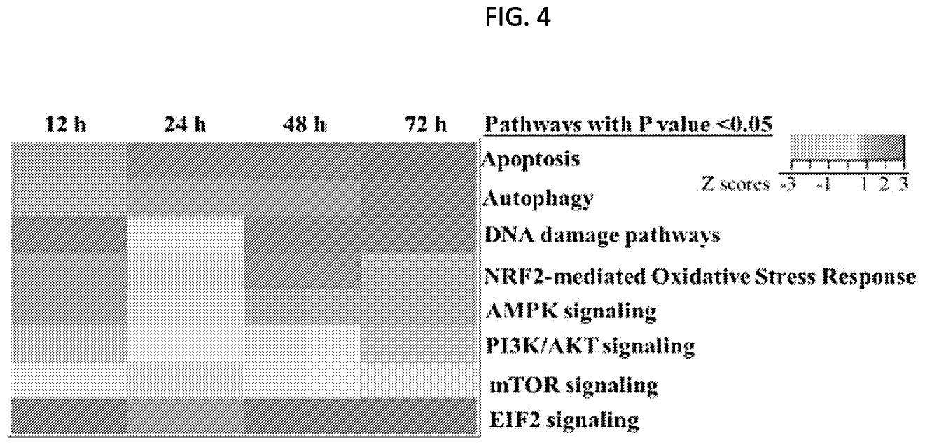

Proteins that were differentially expressed in the TNBC cell line MDA-MB-231 treated with VMY-BC-1 compared to vehicle control were subjected to Ingenuity Pathway Analysis (IPA) to identify significantly altered pathways. Treatment with VMY-BC-1 led to activation of apoptosis, autophagy, and DNA damage pathways, and inhibition of mTOR1, PI3K, and AKT pathways (Z score=0.156, p value=1.97E-10) (FIG. 4).

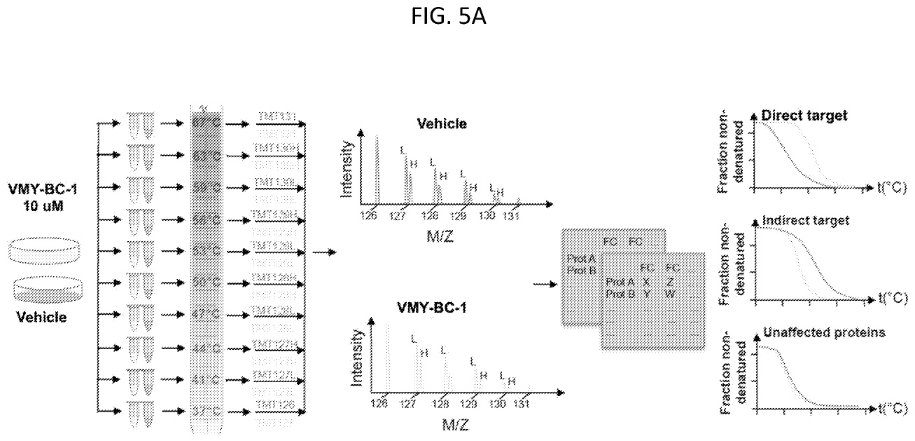

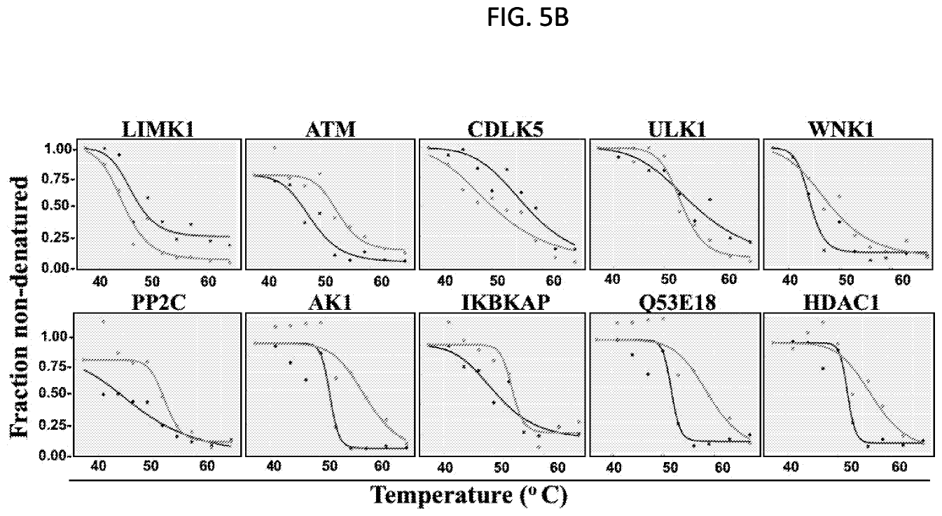

To further elucidate targets that activate the above signaling mechanisms, thermal proteome profiling (TPP) was used to identify binding proteins (Franken et al., Nat. Protoc. 10:1567-1593, 2015; Miettinen et al., EMBO J. 37:e98359, 2018) (FIG. 5A). TPP enables an unbiased assessment of the proteins covalently bound to VMY-BC-1 on a proteome-wide scale. Cell lysates were made from MDA-MB-1 cells treated with vehicle or 10 μM VMY-BC-1 for 24 hours. Lysates were heated at different temperatures for 3 minutes in a thermal cycler. Equal amounts of protein were digested, labeled with TMT 10-plex and analyzed by mass spectrometry. The fold-changes in protein abundances were computed relative to the lowest temperature in the temperature set. After a normalization, melting curves were fitted to the fold-changes of each individual protein. The thermal stability of proteins affected by VMY-BC-1 treatment was calculated using the differences between melting points from control for each protein. From the 5,634 proteins screened, 10 target proteins were identified that bound VMY-BC-1: LIMK1, ATM, CDLK5, ULK1, WNK1, PP2C, AK1, IKBKAP, Q53E18, and HDAC1 (FIG. 5B). The limited fraction of bound protein (0.0017%) suggests a highly selective nature of VMY-BC-1.

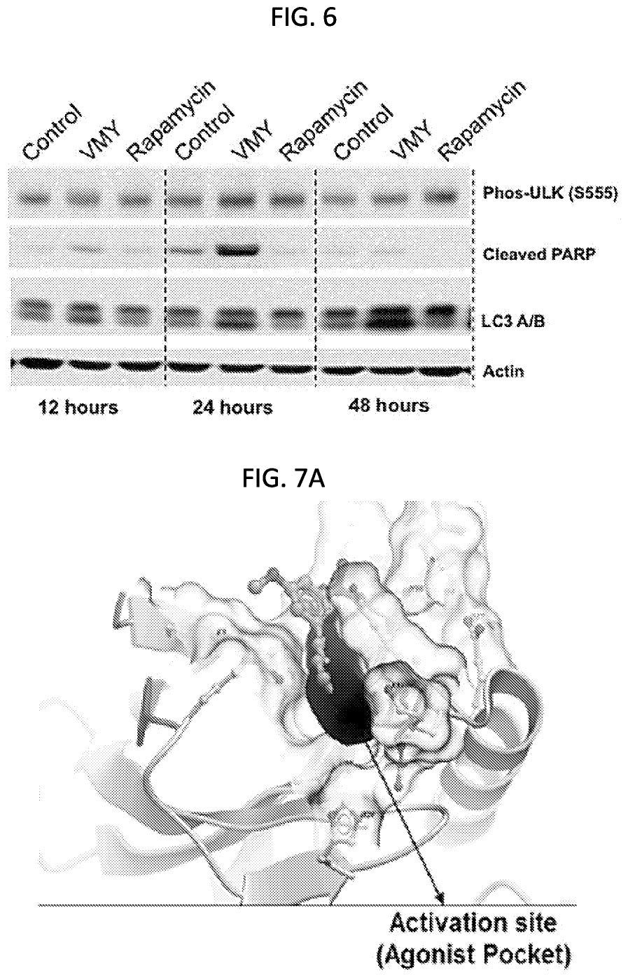

To identify the functional target of VMY-BC-1, preliminary cell viability assays were carried out using siRNA to knock down each of the 10 identified binding proteins. Knock down of both ATM and ULK1 abrogated the functional activity of VMY-BC-1. Considering that that low ULK1 expression is associated with TNBC disease progression (Tang et al., Breast Cancer Res. Treat. 134:549-560, 2012) and autophagy controls the level of various DNA repair and checkpoint proteins in the DNA damage response pathway (Czarny et al., Int. J. Mol. Sci. 16:2641-2662, 2015; Gomes et al., Int. J. Mol. Sci. 18:2351, 2017), ULK1 activity in response to VMY-BC-1 was investigated. Protein extracts were made from MDA-MB-231 cells treated with vehicle control, VMY-BC-1 (10 μM), and Rapamycin (200 nM) at different time points. Western blots of ULK1 and late stage autophagic proteins LC3-I and LC3-II, from VMY-treated TNBC cells, showed increasing amounts of autophagy proteins over time as well as increased phosphorylation of ULK1 and LC3 A/B (FIG. 6). The western blot results also suggested that there was cross talk between the apoptosis and autophagy pathways as observed in the Ingenuity Pathway analysis (FIG. 4).

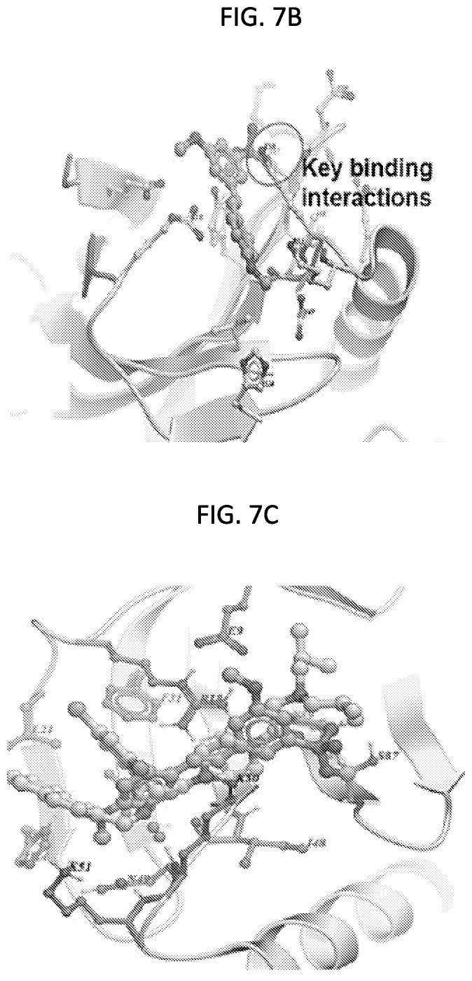

Additionally, an in silico docking model suggests VMY-BC-1 occupies the same binding pocket in the ULK1 agonist site as the ULK1 activator LYN 1604 (FIGS. 7A-7C). Biochemical validation of ULK1 phosphorylation was consistent with data from TMT quantitative proteomics and thermal profiling analysis of pathways affected by VMY-BC-1, and the in silico data supports the hypothesis that VMY-BC-1 activates ULK1 by direct binding and activates autophagy-induced cell death in TNBC.

### Example 4

### Pharmacokinetics Analysis of VMY-BC-1

The parent compound of VMY, resveratrol (RSV), is limited in its utility as a therapeutic agent because of unfavorable pharmacokinetic (PK) properties such as poor bioavailability and rapid and extensive metabolism and excretion (Gambini et al., Oxid. Med. Cell Longev. 2015:837042, 2015; Ko et al., Int. J. Mol. Sci. 18:2589, 2017). Moreover, PK studies of RSV in humans conclude that even high concentration of RSV might be insufficient to achieve in vivo concentrations required for the systemic prevention of cancer. Therefore, in vitro ADME assays were carried out to assess VMY-BC-1 and to identify properties for further optimization.

The ADME results are shown in Table 3. In contrast to resveratrol, VMY-BC-1 was stable in simulated gastric and intestinal fluids, indicating that it may not undergo degradation under the intestinal pH conditions. VMY-BC-1 showed moderate to high intrinsic clearance (43 μL/min/mg of protein) in human liver microsomes (40% remaining at the end of 30 min incubation). VMY-BC-1 did not inhibit major CYP isozymes 2C9, 2C19, 2D6 and 3A with IC₅₀ (the concentration yielding 50% growth inhibition) values in the range of 70-100 μM in the pooled human liver microsomes. VMY-BC-1 weakly inhibited the CYP1A2 isozyme with a IC₅₀ value of 5 μM. VMY-BC-1 showed high permeability in the in vitro Caco-2 cell model, indicating that it may show rapid absorption following oral administration under linear pharmacokinetic conditions. VMY-BC-1 showed moderate plasma protein binding in CD1 mouse plasma.

TABLE 3
In vitro ADME properties of VMY-BC-1

| Assay | VMY-BC-1 | |
|---|---|---|
| Thermodynamic Solubility at pH 7.4 | 0.31 | μM |
| LogD (octanol-water partition coefficient), | 3.37 | |
| pH 7.4 | | |
| Stability in simulated gastric fluid, pH 1.2 | 87.81% remaining | |
| Stability in simulated intestinal fluid | 97.24% remaining | |
| Metabolic stability in human | 41.32 | μL/min/mg protein |
| liver microsome (CLᵢₙₜ) | | |
| IC₅₀ inhibition of CYP1A2 | 4.7 | μM |
| IC₅₀ inhibition of CYP2C9 | 78.4 | μM |
| IC₅₀ inhibition of CYP2C19 | 95.7 | μM |
| IC₅₀ inhibition of CYP2D6 | >100 | μM |
| IC₅₀ inhibition of CYP3A4TST | >100 | μM |
| IC₅₀ inhibition of CYP3A4MD2 | >100 | μM |
| Permeability (Caco-2): A-B | 51 | cm/sec |
| Permeability (Caco-2): B-A- | 20 | cm/sec |
| Efflux (Caco-2) | 0.4 | |

| | |
|---|---|
| Mouse plasma protein binding | 73.48% bound |

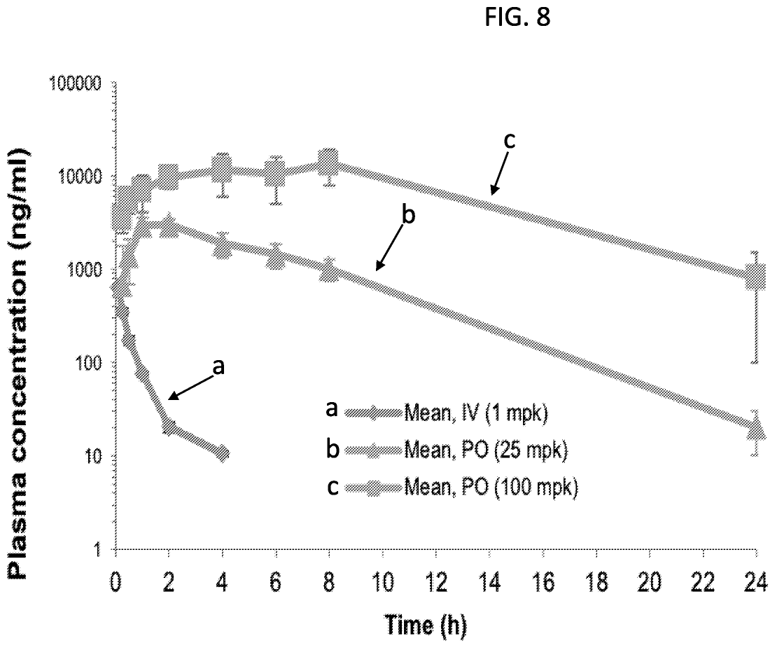

In vivo ADME characteristics were determined in male CD1 mice following VMY-BC-1 administration at 1 mg/kg intravenously and 25 mg/kg and 100 mg/kg orally (FIG. 8). With intravenous administration, VMY-BC-1 showed mono-exponential decay with moderate plasma clearance (50% of hepatic blood flow), with a high volume of distribution (2.4 L/Kg) and an elimination half-life of approximately 1 hour. VMY-BC-1 displayed rapid oral absorption with a time to maximum concentration (Tmax) of 1.0-2.0 hour, and a mean maximum concentration (Cmax) of 3059 ng/mL. Oral suspension bioavailability F (%) was over 100% at 25 mg/kg, indicating elimination was saturated. A 4-fold increase in oral dose (25 to 100 mg/kg) resulted in approximately 5-fold increase in Cmax and 7-fold increase in plasma exposures. Sustained oral absorption following the 100 mg/kg oral administration indicated solubility was not limited oral absorption. When compared to reported PK parameters of resveratrol, VMY-BC-1 had improved PK parameters.

### Example 5

### Efficacy of VMY-BC-1 in Mouse Breast Cancer Xenograft Model

To evaluate the anti-tumor efficacy of VMY-BC-1 in vivo, MDA-MB-231 tumor xenografts were established in the flanks of athymic nude mice. 5×10⁶ MDA-MB-231 TNBC cells in 1×HBSS with 1:1 Matrigel® were implanted subcutaneously in the flank of 5-6 week old female athymic nude mice (CrTac:Ncr-Foxn1^{nu}; Taconic Biosciences, India). Mice (n=8) were treated with vehicle (2% (v/v) Tween® 80+98% (v/v) of 0.5% (w/v) hydroxypropyl methylcellulose (HPMC) in water) or VMY-BC-1 75 mg/kg PO daily for 14 days. The dose volume was 10 mL/kg. Tumor volume, tumor growth inhibition (TGI), body weight, and clinical signs were monitored. Tumor growth was measured twice weekly using a digital Vernier caliper. Tumor volume was calculated as [length×width²]/2. TGI was calculated as:

% TGI={(TV Control_{Final}−TV Control_{Initial})−(TV Treated_{Final}−TV Treated_{Initial})}×100

Tumor volume doubling time (TVDT) was calculated as:

TVDT=(TV Control_{Final}−TV Control_{Initial})

At the end of the study, brain and plasma samples were collected for drug concentration. Tumor tissues were collected for immunohistochemistry, Western blot, and bioanalysis. Statistical analysis of tumor growth in the control and treated groups was performed by Student's T test using GraphPad Prism (Ver. 5.03).

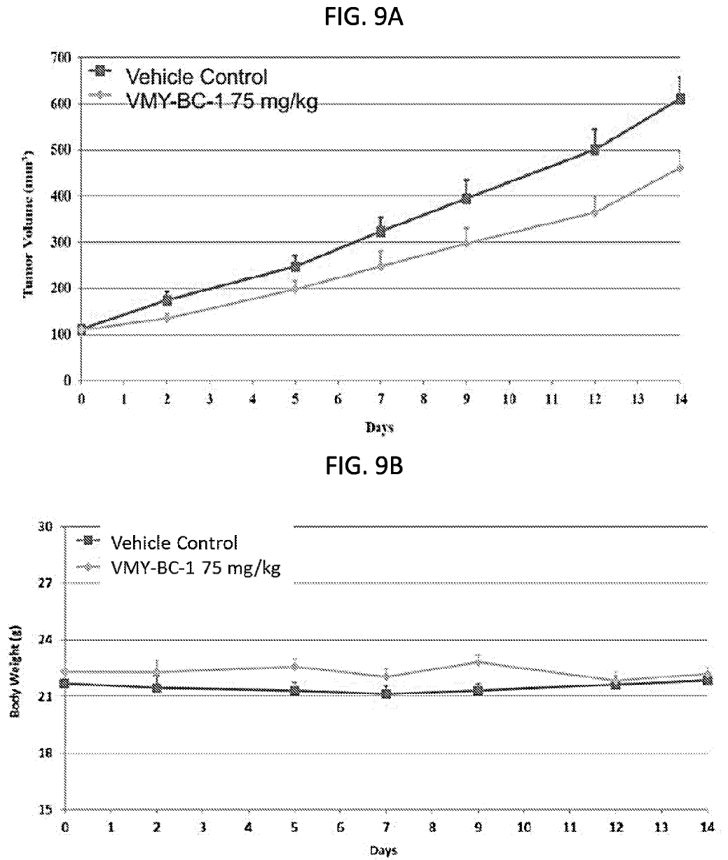

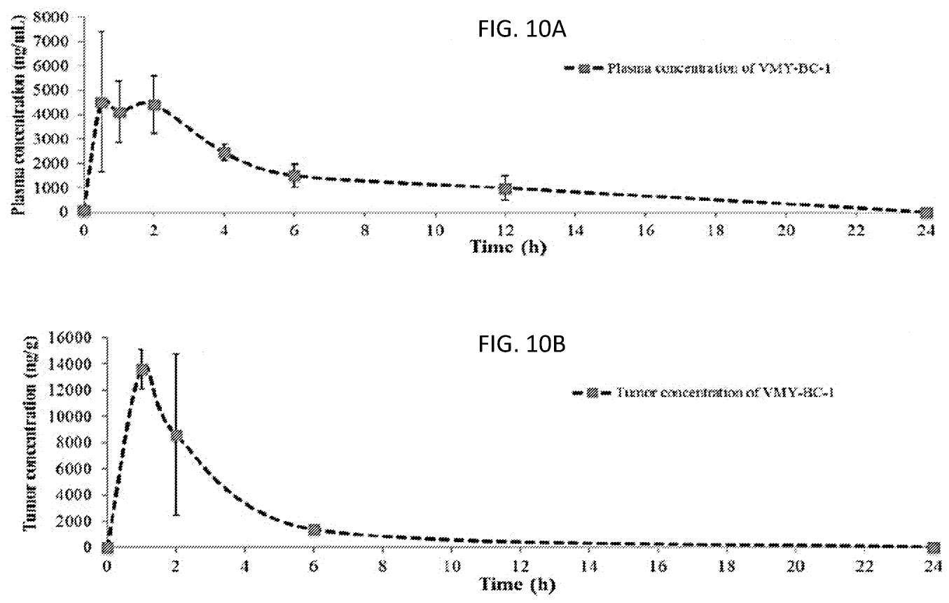

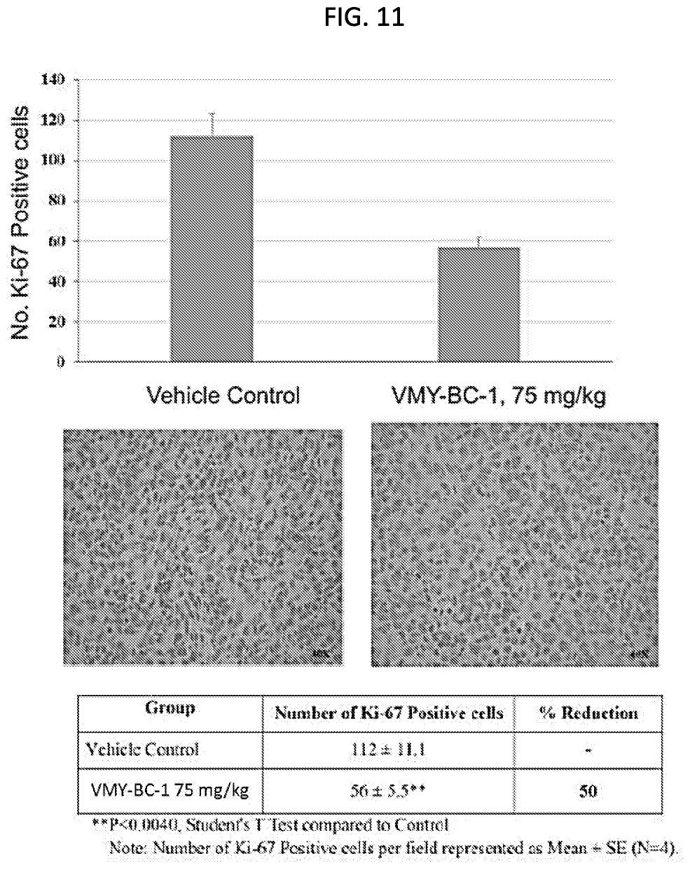

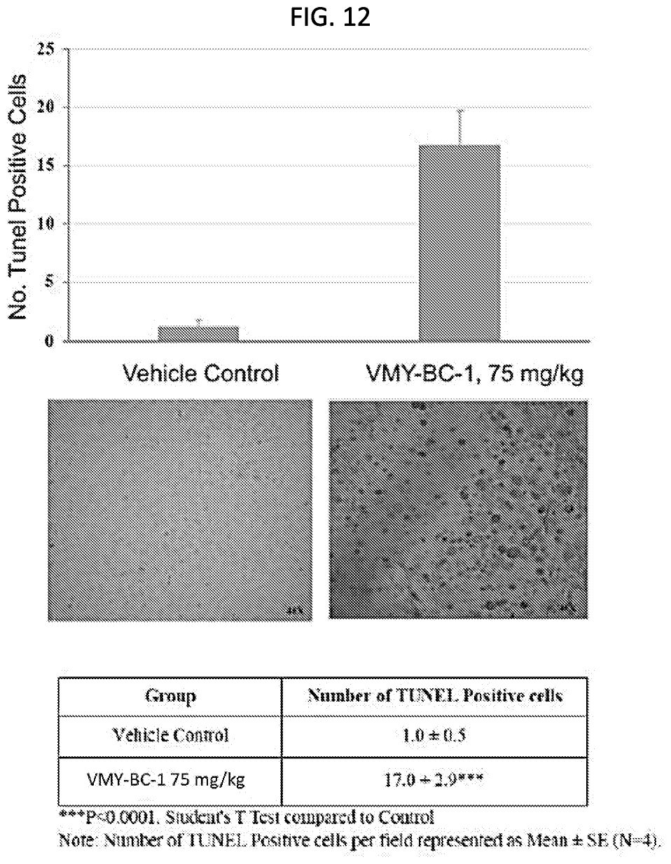

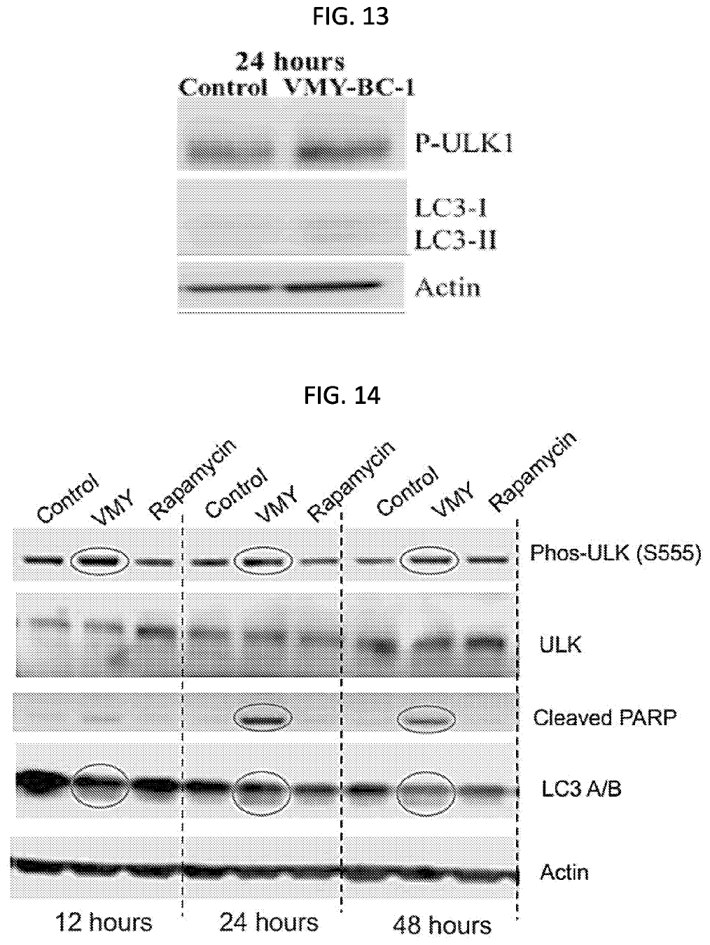

VMY-BC-1 significantly inhibited tumor growth by approximately 30% without any apparent change in body weight (FIGS. 9A and 9B). Analysis of tumor tissue demonstrated higher VMY-BC-1 drug levels in tumor than in plasma (FIGS. 10A and 10B), a 50% reduction in the Ki67 proliferation marker compared to the control treatment (FIG. 11), and a significant increase in apoptosis as measured by TUNEL staining (FIG. 12). The impact of VMY-BC-1 on autophagy proteins in tumor tissue was also investigated. Phospho-ULK1 and LC3 protein expression was upregulated in tumors treated with VMY-BC-1 compared to control (FIG. 13). Finally, as shown in Table 4 and FIGS. 10A and 10B, VMY-BC-1 had 3-fold higher drug concentration (Cₘₐₓ) in tumors than in plasma.

TABLE 4
Plasma vs. tumor VMY-BC-1 PK profile (oral, 75 mg/kg)

| Parameter | Tumor | Plasma |
|---|---|---|
| Cₘₐₓ (ng/g) | 13610.34 | 4529.78 |
| Tₘₐₓ (h) | 1.00 | 0.50 |
| AUCₗₐₛₜ (h * ng/g) | 37936.36 | 32189.04 |
| AUC_{inf} (h * ng/g) | 41005.56 | 32298.04 |
| AUCₑₓₜᵣₐₚ (%) | 7.48 | 0.34 |
| T_{1/2} (h) | 1.52 | 3.15 |
| MRTₗₐₛₜ (h) | 1.94 | 5.76 |

### Example 6

### Effect of VMY-BC-1 on Glioblastoma Cell Lines

The effect of VMY-BC-1 was tested on a panel of GBM cell lines. As shown in Table 5, VMY-BC-1 inhibited growth of established glioma cell lines and patient-derived GBM cell lines compared to normal cell lines. VMY-BC-1 also was more effective at inhibiting cell growth compared to resveratrol. Cells were cultured with VMY-BC-1 or resveratrol for 72 hours and cell viability was assessed using Alamar blue. 50 mM DMSO stocks of both VMY-BC-1 and Resveratrol were used, diluted into cell line media for dose response studies. In this study 100 μM was the highest drug concentration used, with 3-fold dilution for a 10 dose response curve.

TABLE 5
In vitro efficacy of VMY-BC-1 in GBM cell lines

| | IC₅₀ (μM) | |
|---|---|---|
| Cell Line | VMY-BC-1 | Resveratrol |
| Established glioma cell lines | | |
| U251 | 3.98 | 12.00 |
| U87 | 5.88 | 40.00 |
| GL261 | 4.20 | 10.57 |
| Patient-derived cell lines | | |
| GBM4 | 10.99 | 40.58 |
| GBM8 | 9.28 | 26.59 |
| Normal cell lines | | |
| NHA | 26.53 | ND |
| MRCS | 35.3 | ND |

The effect of VMY-BC-1 on autophagy-related proteins in U87 cells was tested. Protein extracts were made from cells treated with vehicle control, VMY-BC-1 (10 μM), or rapamycin (200 nM) for 12-48 hours. Western blots of ULK1 and late stage autophagic proteins LC3-I and LC3-II showed increasing amounts of autophagy proteins over time as well as increased phosphorylation of ULK1 and LC3 A/B (FIG. 14).

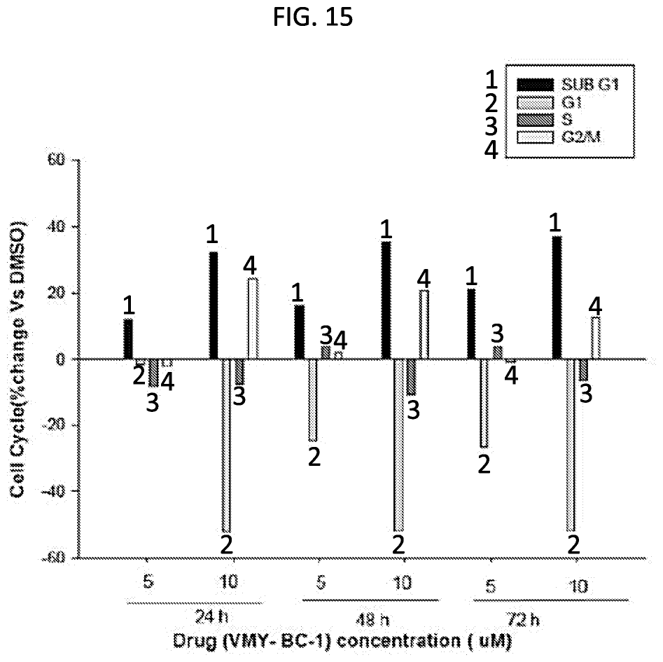

In addition, the effect of VMY-BC-1 on cell cycle pathways in U251 cells was assessed by flow cytometry (FIG. 15). Cells were treated with 5 μM and 10 μM VMY-BC-1 for 24 hours. Cells were trypsinized, centrifuged, and cell pellets were collected. Pellets were resuspended in 1 ml of BS containing 1 mg/ml RNase and 50 mg/ml propidium iodide, incubated in the dark for 30 minutes at room temperature, and analyzed using a FACSort™ Flow Cytometer. The cell cycle distribution was evaluated on DNA plots using the flowzow software.

### Example 7

### Brain Pharmacokinetics of VMY-BC-1

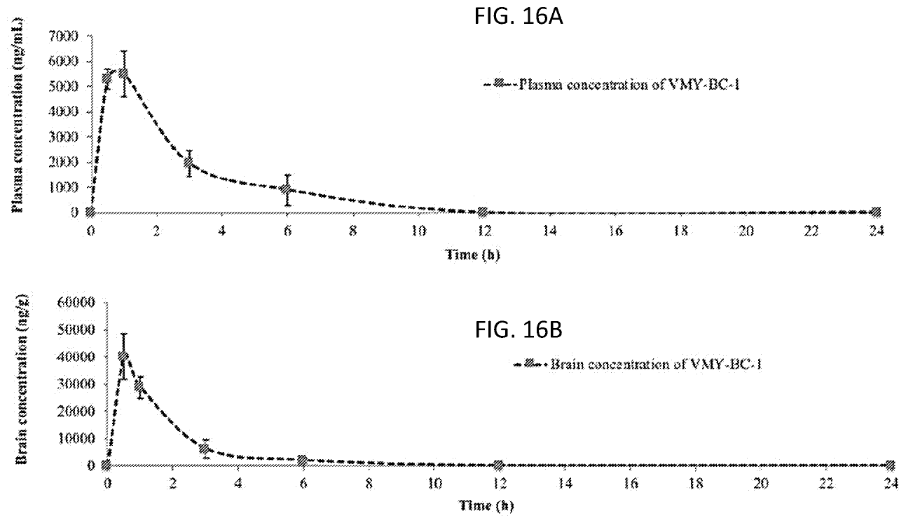

Pharmacokinetics of VMY-BC-1 in the brain were assessed in mice administered 25 mg/kg VMY-BC-1 p.o. (Table 6). VMY-BC-1 had 7-fold higher drug concentration (Cₘₐₓ) in tumors than in plasma (FIGS. 16A and 16B).

TABLE 6
Plasma vs. brain VMY-BC-1 PK profile (oral, 25 mg/kg)

| Parameter | Plasma | Brain |
|---|---|---|
| Cₘₐₓ (ng/g) | 5486.04 | 40017.53 |
| Tₘₐₓ (h) | 1.00 | 0.50 |
| AUCₗₐₛₜ (h * ng/g) | 15692.77 | 74330.76 |
| AUC_{inf} (h * ng/g) | 18194.91 | 78032.82 |
| AUCₑₓₜᵣₐₚ (%) | 13.75 | 4.74 |
| T_{1/2} (h) | 1.95 | 1.32 |
| MRTₗₐₛₜ (h) | 1.96 | 1.48 |

Free drug fraction in brain and plasma compartment was determined in vitro (Table 7). Brain to plasma (B/P) ratio was also determined in vivo (Table 8).

TABLE 7
In vitro binding study

| | Mouse Plasma | | | Mouse Brain Homogenate | | |
|---|---|---|---|---|---|---|
| | | Bound | Recovery | | Bound | Recovery |
| | fu (%) | (%) | (%) | fu (%) | (%) | (%) |
| VMY-BC-1 | 26.52 | 73.48 | 89.32 | 69.92 | 30.08 | 108.02 |

TABLE 8
B/P ratio (VMY-BC-1, 25 mg/kg, single dose, P.O.)

| Time | Mean Plasma | Mean Brain | Brain to Plasma |
|---|---|---|---|
| (h) | Concentration | Concentration | Ratio |
| 0.00 | 0.00 | 0.00 | 0.00 |
| 0.50 | 5273.66 | 40017.53 | 7.59 |
| 1.00 | 5486.04 | 28802.80 | 5.25 |
| 3.00 | 1945.44 | 6158.13 | 3.17 |
| 6.00 | 889.87 | 1948.79 | 2.19 |
| 12.00 | 0.00 | 0.00 | 0.00 |
| 24.00 | 0.00 | 0.00 | 0.00 |

### Example 8

### Efficacy of VMY-BC-1 in Mouse Glioblastoma Xenograft Model

To evaluate the anti-tumor efficacy of VMY-BC-1 in vivo, GBM tumor xenografts were established in the flanks of SCID mice. 5×10⁶ U87 MG (ATCC HTB-14™) cells in 1×HBSS with 1:1 Matrigel® were implanted subcutaneously in the flank of 5-6 week old female SCID mice (C.B-lgh-1b/GbmsTac-Prkdc^{scid}-Lyst^{bg} N7; Taconic Biosciences, India). Mice (n=8) were treated with vehicle (2% (v/v) Tween® 80+98% (v/v) of 0.5% (w/v) hydroxypropyl methylcellulose (HPMC) in water) or VMY-BC-1 75 mg/kg PO daily for 14 days. The dose volume was 10 mL/kg. Tumor volume, tumor growth inhibition (TGI), body weight, and clinical signs were monitored as described in Example 5.

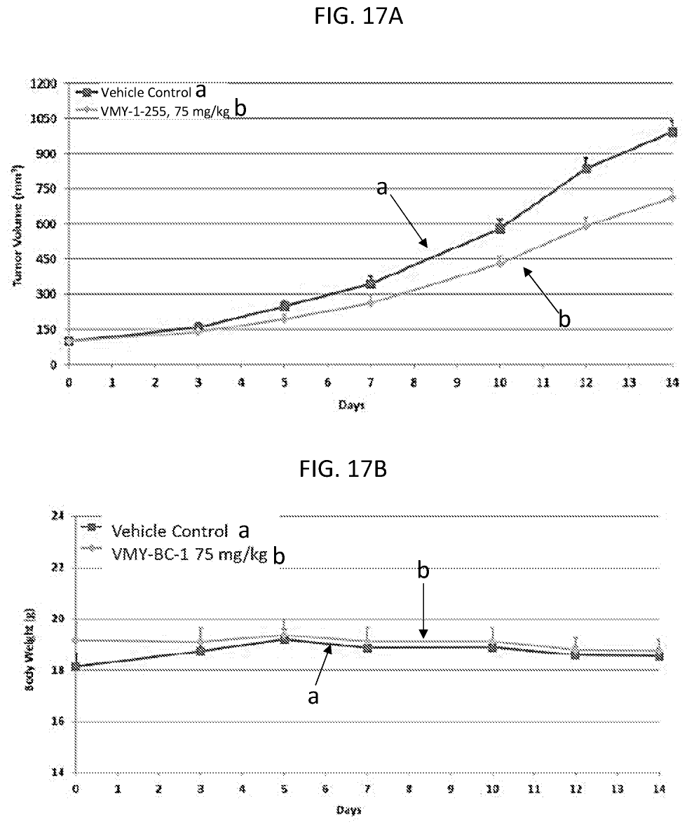

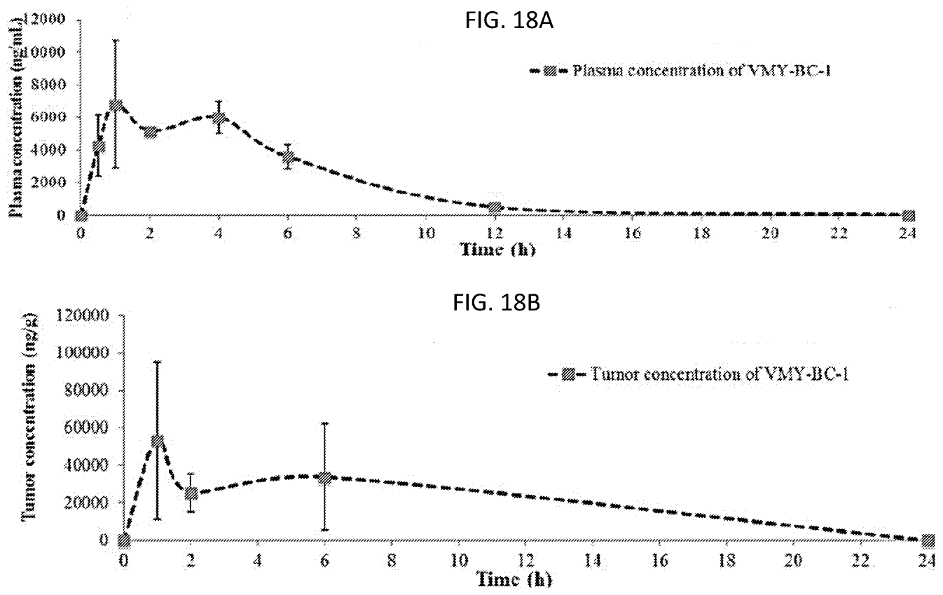

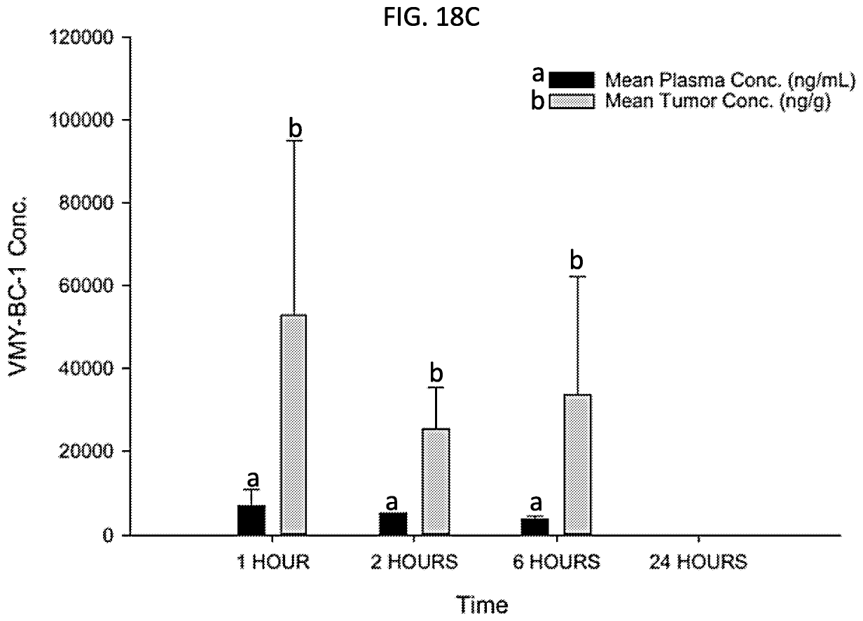

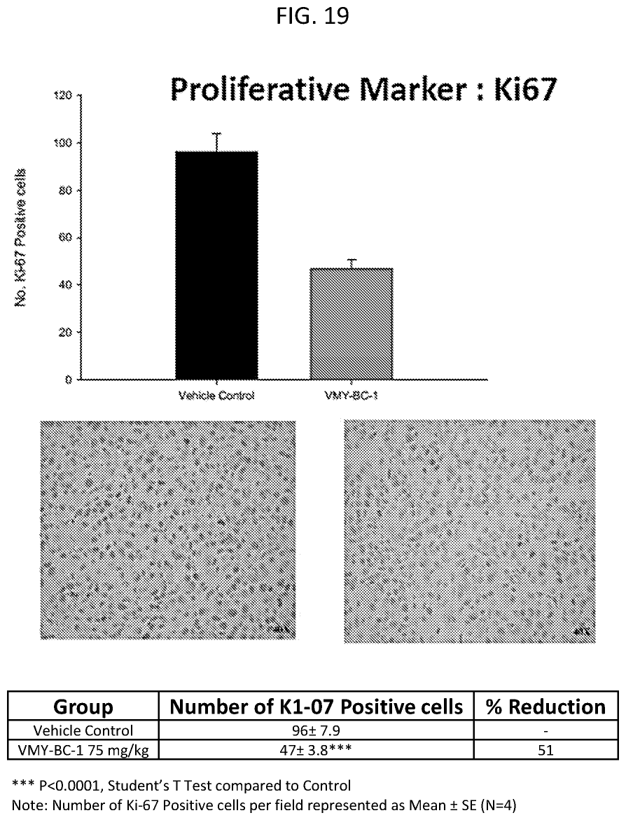

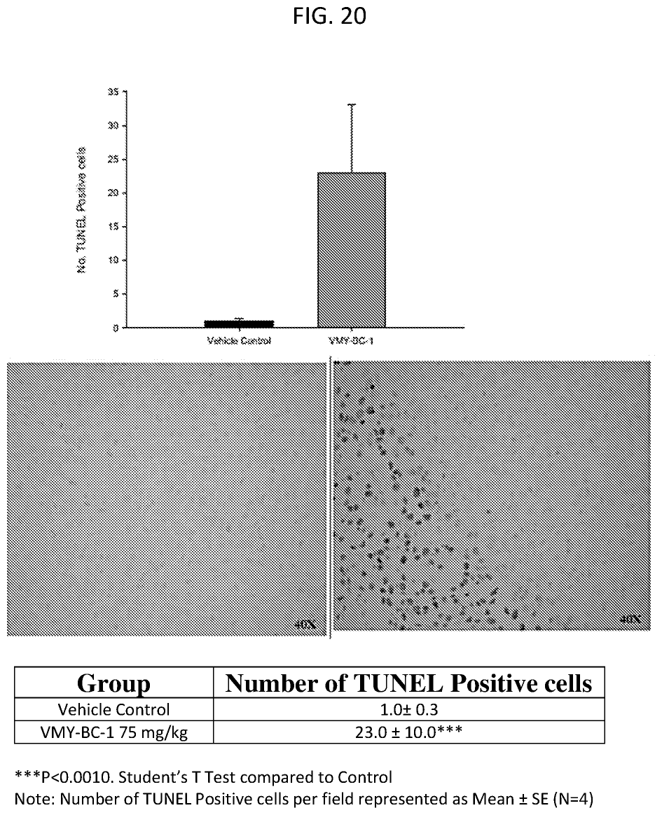

Once daily oral administration of VMY-BC-1 at 75 mg/kg resulted in 32% tumor growth inhibition at the end of 2 weeks (FIG. 17A). No treatment-related adverse effects were observed with respect to body weight (FIG. 17B) and clinical signs. Analysis of tumor tissue demonstrated higher VMY-BC-1 drug levels in tumor than in plasma (FIGS. 18A-18C). In addition, tumor tissue had a 51% reduction in the Ki67 proliferation marker compared to the control treatment (FIG. 19), and a significant increase in apoptosis as measured by TUNEL staining (FIG. 20).

### Example 9

### Efficacy of VMY-BC-1 in C. elegans Huntington's Disease Model

Synchronized populations of wild-type L1 C. elegans hatched overnight were transferred to agar plates followed by VMY-BC-1 was added freshly from a 5 mM stock to the NGM media. Water was used as control. The L4 population of worms was randomly split to control or treatment groups in a density of about 20-30 worms per 6 cm plate dish.

The first day of adulthood was considered day one. In all the experiments with late-onset administration of VMY-BC-1, synchronized L4 animals were moved to the media supplemented with 5′-fluorodeoxyuridine (FUDR, Cayman chemicals) at a final concentration of 1 μM for 2 days to prevent reproduction, then were moved to FUDR-free NGM plates until the final treatments at day 10. The animals that crawled off the plate, ruptured, or died from internal hatching were censored. Worms were transferred to fresh plates every day after reaching adulthood, and every 2 days after reaching 10 days of age. Prodding test was used to count the number of dead worms. Survival curve was plotted using Prism 7 and the significance of the curves calculated by Log-rank (Mantel-Cox) test.

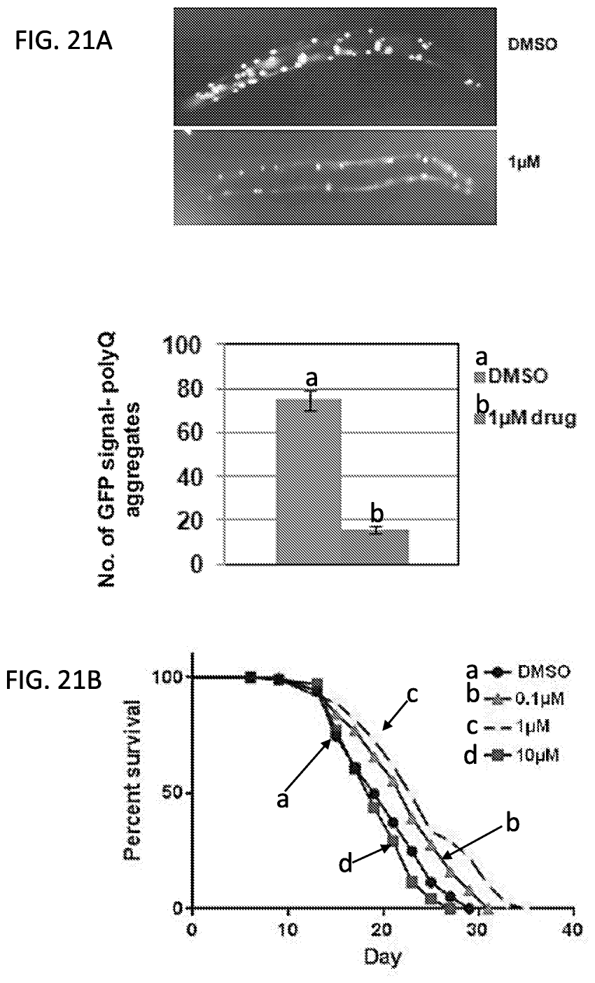

Synchronized populations of polyQ-expressing C. elegans were grown for 72 hours on media containing 1 μM VMY-BC-1. PolyQ aggegrative vacuoles were detected using confocal microscopy. PolyQ aggregations were decreased compared to vehicle (DMSO) (FIG. 21A). Synchronized populations of wild-type worms were grown on media containing VMY-BC-1 from 0.1 to 10 μM. From the results, a dose-dependent lifespan extension was observed (FIG. 21B). Maximal median lifespan extension (˜25%) was observed in animals treated with 1 μM VMY-BC-1.

### Example 10

### Synthesis of VMY-BC-2

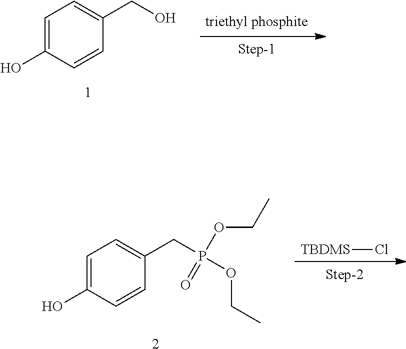

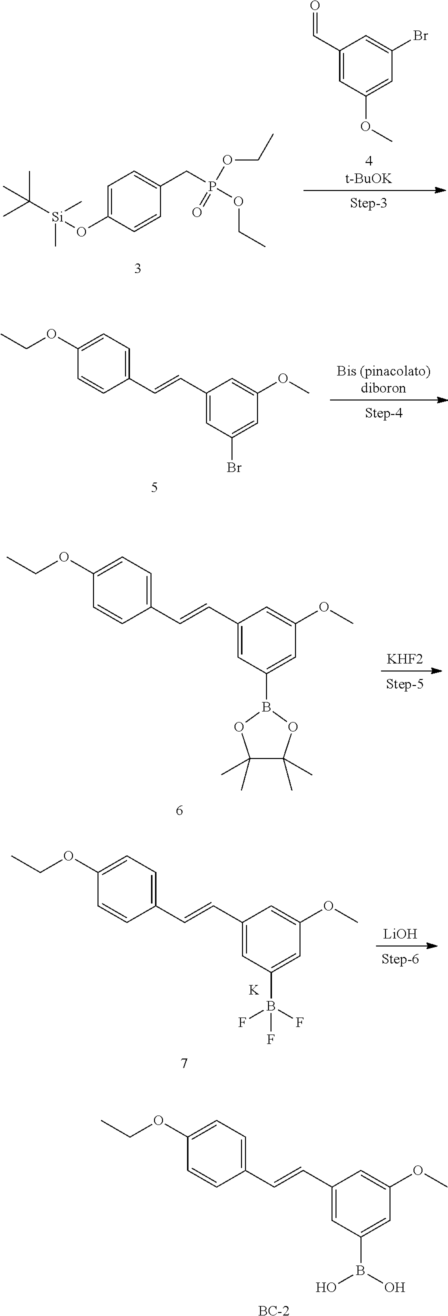

### Step-1: Synthesis of diethyl (4-hydroxybenzyl) phosphonate

In a sealed tube Cpd-1 (10 g) was added triethylphosphite (1.5 eq) and TBAB (0.1 eq) at 25° C. and heated to at 120° C. for 16 h, (progress of the reaction was monitored by TLC) the mixture was cooled to 25° C. Then crude product was purified by column chromatography on silica (ethyl acetate; Hexane 7:3) to yield Cpd 2 (9 gm) as a pale yellow liquid.

### Step-2: Synthesis of diethyl (4-((tert-butyldimethylsilyl) oxy) benzyl) Phosphonate

To a stirred solution of Cpd 2 (5 g) in DCM (100 mL) under nitrogen were added DIPEA (2 eq) at 10° C. to follow by TBDMS-Cl (1.5 eq) stirred for 2 h at 25° C. (progress of the reaction was monitored by TLC). The reaction mixture was washed with H₂O (2×25 mL) and saturated brine solution (25 mL) dried over Na₂SO₄ and concentrated under vacuum to obtained crude compound. Then crude product was purified by column chromatography on silica (ethyl acetate; Hexane 1:1) to yield Cpd-3 (5.5 gm) as a pale yellow liquid.

### Step-3: Synthesis of (E)-1-bromo-3-(4-ethoxystyryl)-5-methoxybenzene

To a stirred solution of Cpd 3 (5 gm) in DMF (2 vol.) were added Cpd 4 (1 eq) at 0° C. follow by t-BuOK (1.1 eq) the mixture was stirred for 30 minutes at 0° C. (progress of the reaction was monitored by TLC). The reaction mixture was poured into ice cold H₂O (100 mL) extracted with EtOAc (2×50 mL), the combined organics were washed with H₂O (2×25 mL) and brine solution (25 mL) dried over Na₂SO₄ and concentrated to obtained crude compound. The crude product was purified by column chromatography on silica (ethyl acetate; Hexane 4:6) to get the Cpd 5 (0.45 g) as a pale yellow liquid.

### Step-4: Synthesis of (E)-2-(3-(4-ethoxystyryl)-5-methoxyphenyl)-4,4,5,5-tetramethyl-1,3,2-dioxaborolane

To a stirred solution of Cpd 5 (0.45 gm) in 1, 4 Dioxan (10 vol.) was added bis(pinacolato) diboron (1.2 eq), KOAc (2 eq) under nitrogen and the reaction mixture was purged with nitrogen for 20 minutes then added PdCl₂(dppf) (0.05 eq) at 25° C. and the mixture was heated to 90° C. for 2 h (progress of the reaction was monitored by TLC). The reaction mixture was cooled to 25° C. filtered through pad of celite washed with EtOAc (20 mL) was evaporated under vacuum. The crude compound was purified by column chromatography on silica (ethyl acetate; Hexane 3:7) to get the Cpd 6 (0.25 gm) as a pale yellow liquid.

### Step-5: Synthesis of E)-(3-(4-ethoxystyryl)-5-methoxyphenyl) Trifluoro-Borane Potassium Salt

To a stirred solution of Cpd 6 (0.25 gm) in MeOH (3 mL) was added aq. KHF2 (6 eq, 4.5M) and the reaction mixture was stirred for 15 min at 25° C. (progress of the reaction was monitored by TLC). The reaction mixture was concentrated in vacuum dissolved in hot acetone filtered and concentrated to obtain Cpd 7 (0.23 gm) as a pale yellow liquid.

### Step-6: Synthesis of (E)-(3-(4-ethoxystyryl)-5-methoxyphenyl) Boronic Acid

To a stirred solution of Cpd 7 (0.23 gm) in MeOH (10 mL) was added aq. LiOH (4 eq, 5 mL) and the reaction mixture was stirred for 2 h at 25° C. (progress of the reaction was monitored by TLC), acidified with 1M HCl extracted with EtOAc (2×10 mL) the combined organics were dried over Na₂SO₄ filtered and concentrated to obtain crude compound. The crude compound was triturated with distilled n-hexane to obtain BC-2 (56 mg) as a pale yellow solid. ¹HNMR: (CD3OD) δ ppm 7.47 (d, 2H, J=9 Hz), 7.30 (s, 1H), 7.12 (m, 2H), 7.01 (m, 2H), 6.90 (d, 2H, J=8.5 Hz), 4.06 (m, 2H), 3.83 (s, 3H), 1.40 (m, 3H).

Mass: m/z 298.99 (M+H).

HPLC: 95.12% at R_{T}=7.64 min.

### Example 11

### Synthesis of VMY-BC-3

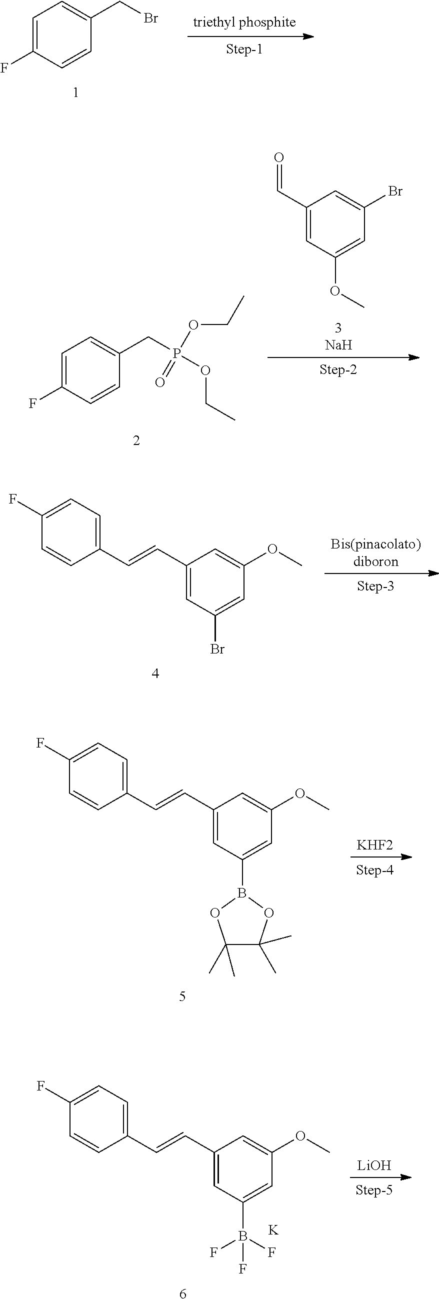

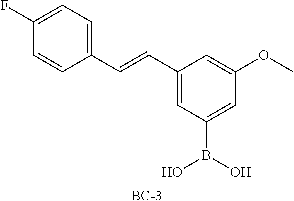 [SMILES: COc1cc(/C=C/c2ccc(F)cc2)cc(B(O)O)c1 ; "BC-3"]

### Step-1: Synthesis of diethyl (4-fluorobenzyl) Phosphonate

In a sealed tube Cpd 1 (2.5 gm) was added triethylphosphite (1.5 eq) at 25° C. and heated to at 80° C. for 16 h (progress of the reaction was monitored by TLC), the reaction mixture was cooled to 25° C., then crude product was purified by column chromatography on silica (ethyl acetate; Hexane 3:7) to yield Cpd 2 (3.8 gm) as a pale yellow liquid.

### Step-2: Synthesis of (E)-1-bromo-3-(4-fluorostyryl)-5-methoxybenzene

To a stirred solution of Cpd 2 (1 gm) in DMF (3 vol.) were added Cpd 3 (1 eq) at 0° C. follow by NaH (1.1 eq) the mixture was stirred for 30 minutes at 0° C. (progress of the reaction was monitored by TLC), the reaction mixture was poured into ice cold H₂O (10 mL) extracted with EtOAc (2×10 mL), the combined organics were washed with H₂O (2×5 mL) and brine solution (10 mL) dried over Na₂SO₄ and concentrated to obtained crude compound. The crude compound was purified by column chromatography on silica (ethyl acetate; Hexane 2:8) to get the Cpd 4 (0.5 g) as a pale yellow liquid.

### Step-3: Synthesis of (E)-2-(3-(4-fluorostyryl)-5-methoxyphenyl)-4,4,5,5-tetramethyl-1,3,2-dioxaborolane

To a stirred solution of Cpd 4 (0.5 gm) in 1,4-dioxane (10 vol.) was added bis pinacolato diborane (1.2 eq), KOAc (2 eq) under nitrogen and the reaction mixture was purged with nitrogen for 20 minutes then added PdCl₂(dppf) (0.05 eq) at 25° C. and the mixture was heated to 90° C. for 2 h. Progress of the reaction was monitored by TLC. The reaction mixture was cooled to 25° C. filtered through pad of celite washed with EtOAc (20 mL) was evaporated under vacuum. The crude was purified by column chromatography on silica (ethyl acetate; Hexane 3:7) to get the Cpd 5 (0.3 gm) as a pale yellow liquid.

### Step-4: Synthesis of (E)-trifluoro (3-(4-fluorostyryl)-5-methoxyphenyl)-borane, Potassium Salt

To a stirred solution of Cpd 6 (0.3 gm) in MeOH (3 mL) was added aq. KHF₂ (6 eq, 4.5M) and the reaction mixture was stirred for 15 min (Progress of the reaction was monitored by TLC), concentrated in vacuum dissolved in hot acetone filtered and concentrated to obtain Cpd 7 (0.2 gm) as a pale yellow liquid.

### Step-5: Synthesis of (E)-trifluoro (3-(4-fluorostyryl)-5-methoxyphenyl)-borane, Potassium Salt

To a stirred solution of Cpd 7 (0.2 gm) in MeOH (10 mL) was added aq. LiOH (4 eq, 2 mL) and the reaction mixture was stirred for 2 h at 25° C. (Progress of the reaction was monitored by TLC), acidified with 1M HCl extracted with EtOAc (2×10 mL) the combined organics were washed with H₂O (2×5 mL) and brine solution (10 mL) dried over Na₂SO₄ filtered and concentrated to obtain crude compound. The crude compound was triturated with distilled n-hexane to obtain BC-3 (55 mg) as a white solid.

¹HNMR: (CD3OD) δ ppm 7.58 (m, 2H), 7.32 (s, 1H), 7.17 (m, 5H), 4.85 (s, 3H)

Mass: m/z 271.28 (M−H).

HPLC: 96.40% at R_{T}=7.53 min.

### Example 12

### Synthesis of BC-4 and BC-5 Precursor CPD-09

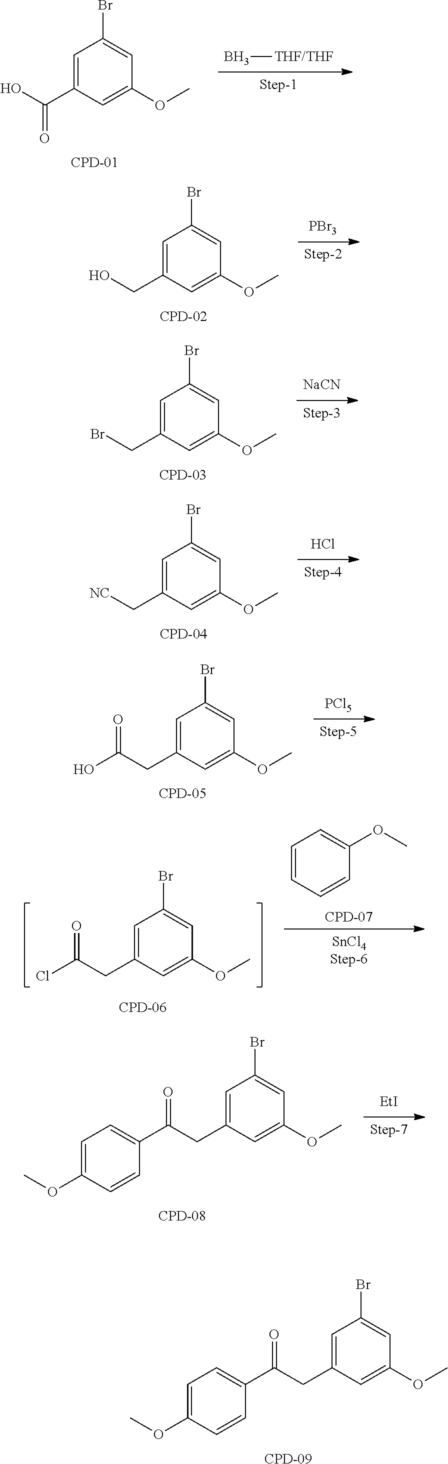

### Step-1:

In THF (250.0 mL) solution of 3-bromo-5-methoxybenzoic acid (25.0 g, 0.109 mol), at 0-5° C. Borane-THF was added (326.1 mL, 0.326 mol), and allowed the reaction mass at 25±5° C. for 16 hours. Reaction completion was monitored by TLC. After reaction completion the reaction mixture was quenched with sodium bicarbonate solution (125.0 ml) and extracted with MTBE (125.0 mL). Organic phase was washed with water (125.0 mL) and saturated brine (50.0 mL), the organic layer was dried over Na₂SO₄, the solvent was distilled off under reduced pressure to get CPD-02 off white solid (17.52 g 74.5%).

### Step 2:

To a stirred solution of CPD-02 in DCM (17.5 g, 0.081 mol), at 0-5° C. phosphorus bromide was added (10.9 g, 0.041 mol), and stirred the reaction mass at 25±5° C. for 2 hours. Reaction completion was monitored by TLC. After reaction completion the reaction mixture was quenched with sodium bicarbonate solution (35.0 mL) and separated the layer. Organic phase was washed with water (35.0 mL) and saturated brine (35.0 mL), the organic layer was dried over Na₂SO₄, the solvent was distilled off under reduced pressure to get the CPD-03 as white solid (17.52 g, 77.7%).

### Step 3:

In DMSO (175 mL) solution of CPD-03 (17.5 g, 0.063 mol), at room temperature sodium cyanide (3.68 g, 0.075 mol) was added and warm the reaction mass at 45° C. for 4 h. Reaction completion was monitored by TLC. After reaction completion the reaction mixture was quenched with sodium bicarbonate solution (175.0 mL) and extracted with ethyl acetate (175.0 mL). Organic phase was washed with water (2*35.0 mL) and saturated brine (35.0 mL), the organic layer was dried over Na₂SO₄, the solvent was distilled off under reduced pressure to get the CPD-04 as brown gummy mass (11.3 g, 79.9%).

### Step 4:

The CPD-04 (11.2 g, 0.050 mol) was heated with concentrated hydrochloric acid (112.0 mL) at 95±5° C. for 16 h. Reaction completion was monitored by TLC. After reaction completion the reaction mixture was cooled at 25° C. and extracted with MTBE (112.0 mL). Organic phase was washed with water (22.4 mL) and saturated brine (22.4 mL), the organic layer was dried over Na₂SO₄, the solvent was distilled off under reduced pressure. Crude was purified by column chromatography to get CPD-05 as off white solid (7.3 g, 60.1%).

### Step 5&6:

Phosphorous pentachlorides (7.2 g, 0.038 mol) was added in portions to a solution of CPD-05 (7.2 g, 0.030 mol) in DCE (72.0 mL). The mixture was refluxed for 30 min, then cooled to 0° C., anisole (14.3 g, 0.133 mol) was added slowly, followed by the dropwise addition of anhydrous stannic chloride (19.2 g, 0.073 mol) solution in DCE (72.0 mL). Stirred the reaction mass at 25±5° C. for 16 h. Reaction completion was monitored by TLC. After reaction completion the deep red solution was poured into ice (72.0 g) and concentrated HCl (36.0 mL). Product was extracted with ethyl acetate (72.0 mL) and washed the organic layer with 5% aqueous NaHCO₃ (15.0 mL) followed by saturated brine (15.0 mL), the organic layer was dried over Na₂SO₄, the solvent was distilled off under reduced pressure. Crude was purified by column chromatography to get CPD-08 as off white to pale pink solid (5.93 g 60.0%).

### Step 7:

Sodium hydride (0.72 g of a 57-63% dispersion in mineral oil, 0.018 mol) was washed twice with dry THF (58.0 mL) and then added slowly as a slurry in THF to a stirred solution of 10.0 g CPD-08 (5.8 g, 0.017 mol) in THF (58.0 mL). After the addition was completed, ethyl iodide (2.8 g, 0.018 mol) was added dropwise, and heated the reaction mass at 50±5° C. for 16 h. Reaction completion was monitored by TLC. After reaction completion the reaction mass was cooled to 25±5° C. and extracted with MTBE (58.0 mL). Washed the organic layer with 5% aqueous NaHC0₃ (15.0 mL) followed by saturated brine (15.0 mL). The organic layer was dried over Na₂SO₄ and solvent was distilled off under reduced pressure to get CPD-09 as off pale yellow gummy mass (6.16 g, 98.1%).

### Example 13

### Synthesis of VMY-BC-4

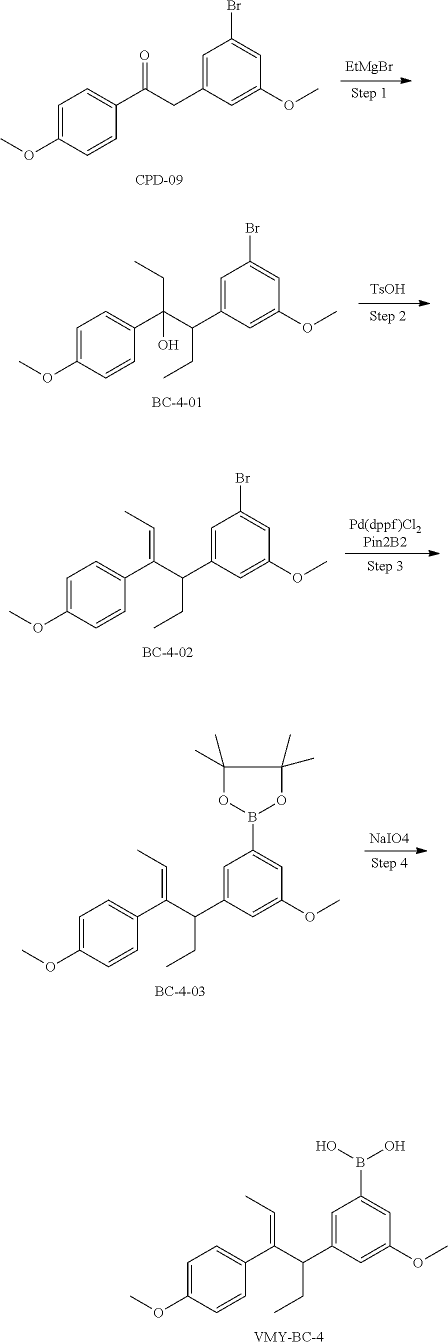

### Steps 1 and 2:

To a solution of CPD-09 (3.0 g, 0.008 mol) in THE (30.0 mL) at 25° C., ethyl magnesium iodide 1M in THE (41.2 mL, 0.041 mol) was added dropwise and heated the reaction mass to 45° C. for 4 h. Reaction completion was monitored by TLC. After reaction completion cooled the reaction mass and quenched with aq. Ammonium chloride solution (30.0 ml) and extracted with MTBE (30.0 mL). Washed the organic layer with 5% aqueous NaHCO₃ (15.0 mL) followed by saturated brine (15.0 mL). The organic layer was dried over Na₂SO₄ and solvent was distilled off under reduced pressure to get BC-4-01 as off pale yellow gummy mass.

Diluted the gummy mass with DCM (30.0 mL) and added p-toluene sulfonic acid (0.2 g, 0.008 mol). Warmed the reaction mass to 45° C. for 4 h. Reaction completion was monitored by TLC. After reaction completion cooled the reaction mass to 25° C. After reaction completion cooled the reaction mass and quenched with water (30.0 ml) and separated the layers. Washed the organic layer with 5% aqueous NaHCO₃ (15.0 mL) followed by saturated brine (15.0 mL). The organic layer was dried over Na₂SO₄ and solvent was distilled off under reduced pressure. Crude was purified by column chromatography to get BC-4-02 (Exo) as pale yellow gummy mass (1.8 g, 58.1%).

### Steps 3 and 4:

To a stirred solution of BC-4-02 (Exo) (1.3 g, 0.003 mol;) in DMSO (13 mL), was added KOAc (0.7 g, 0.007 mol) followed by bis(pinacolato)diboron (1.1 g, 0.004 mol) and purged with argon for 15 min at 35±5° C. To that, was added Pd(dppf)Cl₂ (0.05 g, 0.0001 mol) under argon atmosphere and stirred at 100° C. for 3 h. Reaction completion was monitored by TLC. Upon reaction completion, RM was filtered through celite and washed with EtOAc (13 mL). Filtrate was washed with water (13.0 mL), dried over Na₂SO₄ and concentrated. Crude as such dissolved in THE (13.0 mL), was added NaIO₄ (3.72 g, 0.017 mol) at 25±5° C. and stirred for 8 h. Reaction completion was monitored by TLC. Diluted the reaction mass with EtOAc: 1N HCl (7.8 mL: 2.6 mL) and stirred for 30 min. Separated the layers, organic phase was washed with saturated aq. NaHCO₃ followed by brine solution. Organic phase was dried over Na₂SO₄ and concentrated. Crude was purified by prep-HPLC followed by slurried in n-Hexane, decanted the top layer and dried the residue under high vacuum for 2 h to get VMY-BC-4 (Exo) as brown gummy mass (35 mg, 3.0%).

### Example 14

### Synthesis of VMY-BC-5

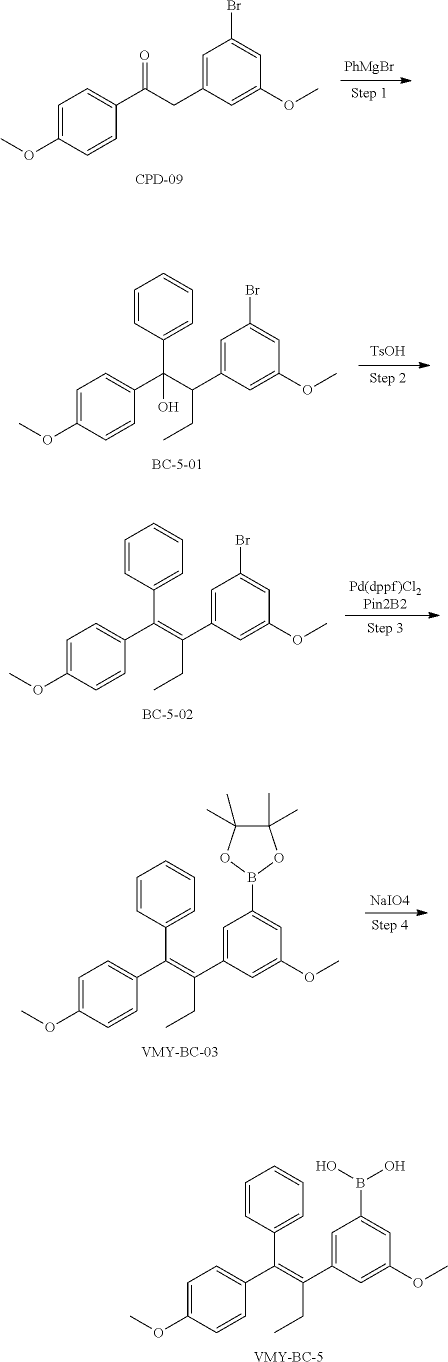

### Steps 1 and 2:

To a solution of CPD-09 (3.0 g, 0.008 mol) in THE (30.0 mL) at 25° C., phenyl magnesium iodide 1M in THE (41.2 mL, 0.041 mol) was added dropwise and heated the reaction mass to 45° C. for 4 h. Reaction completion was monitored by TLC. After reaction completion cooled the reaction mass and quenched with aq. Ammonium chloride solution (30.0 ml) and extracted with MTBE (30.0 mL). Washed the organic layer with 5% aqueous NaHCO₃ (15.0 mL) followed by saturated brine (15.0 mL). The organic layer was dried over Na₂SO₄ and solvent was distilled off under reduced pressure to get BC-5-01 as off pale yellow gummy mass.

Diluted the gummy mass with DCM (30.0 mL) and added p-toluene sulfonic acid (0.2 g, 0.001 mol). Warmed the reaction mass to 45° C. for 4 h. Reaction completion was monitored by TLC. After reaction completion cooled the reaction mass to 25° C. After reaction completion cooled the reaction mass and quenched with water (30.0 ml) and separated the layers. Washed the organic layer with 5% aqueous NaHC03 (15.0 mL) followed by saturated brine (15.0 mL). The organic layer was dried over Na₂SO₄ and solvent was distilled off under reduced pressure. Crude was purified by column chromatography to get BC-5-02 as pale yellow gummy mass (1.3 g, 37.3%).

### Steps 3 and 4:

To a stirred solution of BC-5-02 (1.2 g, 0.002 mol) in DMSO (12.0 mL), was added KOAc (0.6 g, 0.006 mol) followed by bis(pinacolato)diboron (0.86 g, 0.003 mol) and purged with argon for 15 min at 35±5° C. To that, was added Pd(dppf)Cl₂ (0.04 g, 0.0001 mol) under argon atmosphere and stirred at 100° C. for 3 h. Reaction completion was monitored by TLC. Upon reaction completion, RM was filtered through celite and washed with EtOAc (12.0 mL). Filtrate was washed with water (12.0 mL), dried over Na₂SO₄ and concentrated. Crude as such dissolved in THE (12.0 mL), was added NaIO₄ (3.1 g, 0.014 mol) at 25±5° C. and stirred for 8 h. Reaction completion was monitored by TLC. Diluted the reaction mass with EtOAc: 1N HCl (7.2 mL: 1.4 mL) and stirred for 30 min. Separated the layers, organic phase was washed with saturated aq. NaHCO₃ followed by brine solution. Organic phase was dried over Na₂SO₄ and concentrated. Crude was purified by prep-HPLC followed by slurried in n-Hexane and solid was filtered and dried under high vacuum for 2 h to get VMY-BC-5 as white solid (44 mg, 4.1%).

### Example 15

### Pharmacokinetics of BC-2 and BC-3

Pharmacokinetics of BC-2 and BC-3 were compared to BC-1 and resveratrol. Pharmacokinetics were assessed in mice administered a single oral dose of 5 mg/kg of the indicated compound (Tables 9 and 10). Each of BC-1, BC-2, and BC-3 had higher concentration in brain than in plasma. RSV was below quantification level (BQL) at all time points.

TABLE 9
BC analog drug levels in mouse plasma and brain

| Time | Plasma (ng/ml), Mean (4) ± SE | | | | Brain (ng/g), Mean (4) ± SE | | | |
|---|---|---|---|---|---|---|---|---|
| (min) | BC-1 | BC-2 | BC-3 | RSV | BC-1 | BC-2 | BC-3 | RSV |
| 0 | BQL | BQL | BQL | BQL | BQL | BQL | BQL | BQL |
| 30 | 578 ± 55 | 841 ± 45 | 439 ± 67 | BQL | 1558 ± 237 | 3425 ± 430 | 1951 ± 323 | BQL |
| 60 | 470 ± 61 | 732 ± 56 | 217 ± 28 | BQL | 1405 ± 211 | 4136 ± 23 | 1233 ± 219 | BQL |
| 180 | 60 ± 48 | 281 ± 46 | 17 ± 10 | BQL | 169 ± 58 | 983 ± 193 | 95 ± 26 | BQL |
| 360 | BQL | 43 ± 5 | BQL | BQL | BQL | 94 ± 15 | BQL | BQL |
| 900 | BQL | BQL | BQL | BQL | BQL | BQL | BQL | BQL |
| 1440 | BQL | BQL | BQL | BQL | BQL | BQL | BQL | BQL |

TABLE 10
Selected pharmacokinetic parameters of BC analogs

| | Plasma (ng/ml), Mean (4) ± SE | | | Brain (ng/g), Mean (4) ± SE | | |
|---|---|---|---|---|---|---|
| Parameters | BC-1 | BC-2 | BC-3 | BC-1 | BC-2 | BC-3 |
| Tₘₐₓ(min) | 30 | 30 | 30 | 30 | 60 | 30 |
| Cₘₐₓ(ng/ml) | 578 | 841 | 439 | 1560 | 4140 | 1950 |
| AUC₍₀₋ₜ₎ | 48200 | 115000 | 25500 | 138000 | 496000 | 129000 |
| (min * ng/ml) | | | | | | |
| AUC_{(inf)} | 51900 | 120000 | 26300 | 149000 | 504000 | 134000 |
| (min * ng/ml) | | | | | | |
| T½ | 44.0 | 72.8 | 32.3 | 44.3 | 54.8 | 33.8 |
| K_{d} (Lambda Z), | 0.0157 | 0.00952 | 0.0215 | 0.0156 | 0.0127 | 0.0205 |
| min⁻¹ | | | | | | |

### Example 16

### Pharmacokinetics of BC-4 and BC-5

Pharmacokinetics of BC-4 and BC-5 were compared to BC-1. Pharmacokinetics were assessed in Balb/c mice administered a single oral dose of 5 mg/kg of the indicated compound (Tables 11 and 12). Both BC-4 and BC—S had higher concentrations in plasma than brain.

TABLE 11
Pharmacokinetics profile of BC analogs in mouse plasma and brain

| Time | Plasma (ng,/ml), Mean (4) ± SE | | | Brain (ng/g), Mean (4) ± SE | | |
|---|---|---|---|---|---|---|
| (min) | BC-1 | BC-4 | BC-5 | BC-1 | BC-4 | BC-5 |
| 0 | BQL | BQL | BQL | BQL | BQL | BQL |
| 30 | 578 ± 55 | 255 ± 20 | 1599 ± 40 | 1558 ± 237 | 164 ± 16 | 142 ± 32 |
| 60 | 470 ± 61 | 163 ± 20 | 1524 ± 106 | 1405 ± 211 | 139 ± 14 | 205 ± 39 |
| 180 | 60 ± 48 | 36 ± 6 | 742 ± 91 | 169 ± 58 | BQL | 105 ± 26 |
| 360 | BQL | 22 ± 3 | 290 ± 35 | BQL | BQL | 28 ± 6 |
| 900 | BQL | BQL | 49 ± 10 | BQL | BQL | BQL |
| 1440 | BQL | BQL | 15 ± 9 | BQL | BQL | BQL |

TABLE 12
Selected pharmacokinetics parameters of BC analogs

| | Plasma (ng/ml), Mean (4) ± SE | | | Brain (ng/g), Mean (4) ± SE | | |
|---|---|---|---|---|---|---|
| Parameters | BC-1 | BC-4 | BC-5 | BC-1 | BC-4 | BC-5 |
| Tₘₐₓ (min) | 30 | 30 | 30 | 30 | 30 | 60 |
| Cₘₐₓ (ng/ml) | 578 | 255 | 1600 | 1560 | 164 | 205 |
| AUC₍₀₋ₜ₎ | 48200 | 25300 | 376000 | 138000 | 6990 | 35700 |
| (min * ng/ml) | | | | | | |
| AUC_{(inf)} | 51900 | 28300 | 381000 | 149000 | 32400 | 39800 |
| (min * ng/ml) | | | | | | |
| T½ | 44.0 | 94.5 | 251 | 44.3 | 127 | 103 |
| K_{d} (Lambda Z), | 0.0157 | 0.00734 | 0.00276 | 0.0156 | 0.00546 | 0.00672 |
| min⁻¹ | | | | | | |

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1. A compound selected from: 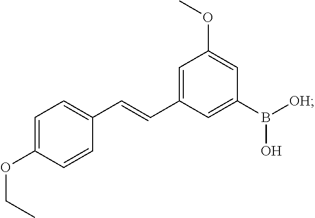 [SMILES: CCOc1ccc(/C=C/c2cc(OC)cc(B(O)O)c2)cc1]
or a compound having a structure of Formula II, IIA, or IIB: 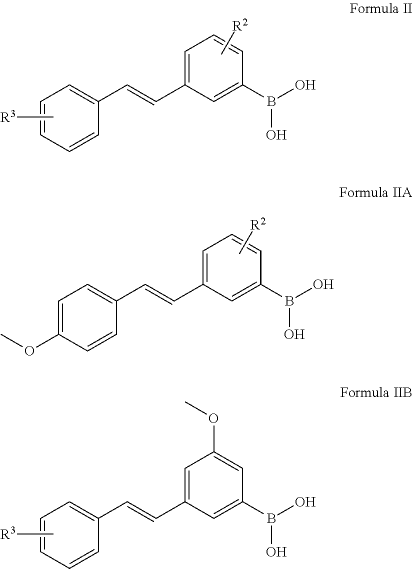
or a pharmaceutically acceptable salt, stereoisomer, tautomer, or prodrug thereof, wherein
each of R² and R³ independently are selected from aliphatic, haloaliphatic, halogen, —C(O)OR^{b}, —C(O)N(R^{b})₂, —C(O)H, —OC(O)R^{b}, —OC(O)OR^{b}, —NR^{b}S(O)₂R^{b}, —S(O)₂N(R^{b})₂, —[(C(R^{b})₂)ₙO]ₘR^{b}, —OR^{b}, —SR^{b}, —N(R^{b})₂, wherein each R^{b} independently is hydrogen, aliphatic, heteroaliphatic, haloaliphatic, aromatic, or combinations thereof; and
n and m independently are integers ranging from 1 to 50.

2. The compound of claim 1, wherein each R^{b} independently is hydrogen, alkyl, alkenyl, alkynyl, heteroalkyl, heteroalkenyl, heteroalkynyl, haloalkyl, haloalkenyl, haloalkynyl, aryl, heteroaryl, or combinations thereof.

3. The compound of claim 1, wherein each of R² and R³ independently is selected from alkyl, —C(H)(halogen)₂, —C(H₂)(halogen), —C(halogen)₃, Cl, F, Br, I, —C(O)OH, —C(O)NH₂, —C(O)N(H)alkyl, —C(O)N(alkyl)₂, —C(O)O-alkyl, —OC(O)-alkyl, —OC(O)O-alkyl, —NHS(O)₂alkyl, —N(alkyl)S(O)₂alkyl, —S(O)₂NH₂, —S(O)₂N(alkyl)₂, —S(O)₂N(H)alkyl, [—(CH₂)ₙO—]ₘH, —OH, O-alkyl, —O-heteroalkyl, —SH, —S-alkyl, —S-heteroalkyl, —NH₂, —N(alkyl)₂, or —N(H)alkyl, wherein each alkyl group independently is selected from lower alkyl and wherein the heteroalkyl group is —(CH₂)_{q}N(alkyl)₂, wherein q is an integer selected from 1, 2, or 3.

4. The compound of claim 3, wherein each of R² and R³ independently is selected from methyl, ethyl, propyl, isopropyl, butyl, isobutyl, t-butyl, Cl, F, —CF₃, —COOH, —OC(O)Me, —C(O)NH₂, NH₂, —NMe₂, —NHMe, —SO₂NH₂, —OEt, —O(CH₂)₂N(Me)₂, or —OiPr.

5. The compound of claim 1, wherein the compound is any one of

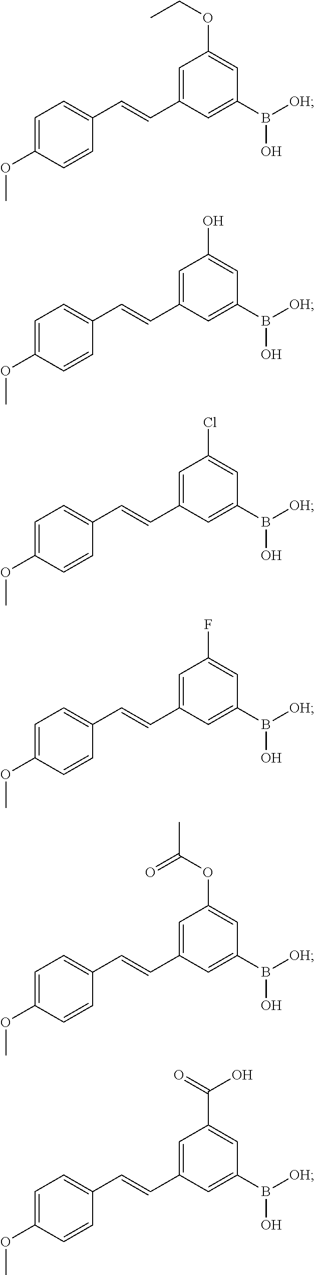 [SMILES: CCOc1cc(/C=C/c2ccc(OC)cc2)cc(B(O)O)c1 ; COc1ccc(/C=C/c2cc(O)cc(B(O)O)c2)cc1 ; COc1ccc(/C=C/c2cc(Cl)cc(B(O)O)c2)cc1 ; COc1ccc(/C=C/c2cc(F)cc(B(O)O)c2)cc1 ; COc1ccc(/C=C/c2cc(OC(C)=O)cc(B(O)O)c2)cc1 ; COc1ccc(/C=C/c2cc(B(O)O)cc(C(=O)O)c2)cc1]

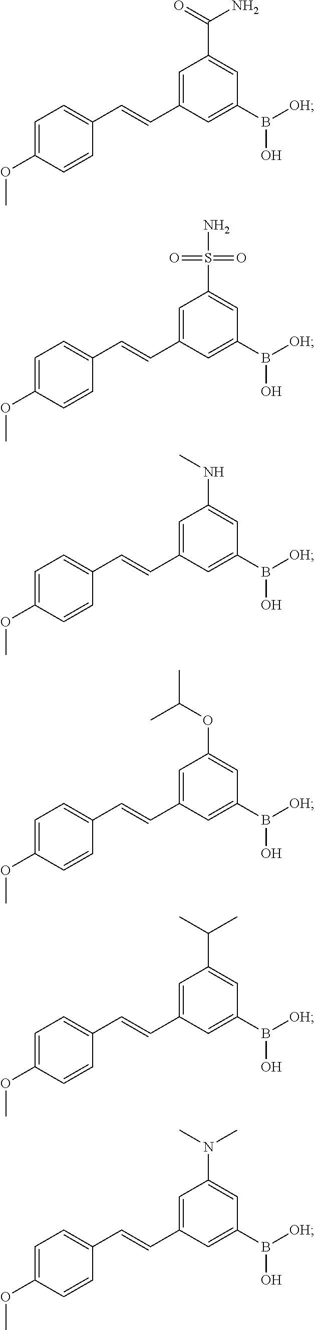 [SMILES: COc1ccc(/C=C/c2cc(B(O)O)cc(C(N)=O)c2)cc1 ; COc1ccc(/C=C/c2cc(B(O)O)cc(S(N)(=O)=O)c2)cc1 ; CNc1cc(/C=C/c2ccc(OC)cc2)cc(B(O)O)c1 ; COc1ccc(/C=C/c2cc(OC(C)C)cc(B(O)O)c2)cc1 ; COc1ccc(/C=C/c2cc(B(O)O)cc(C(C)C)c2)cc1 ; COc1ccc(/C=C/c2cc(B(O)O)cc(N(C)C)c2)cc1]

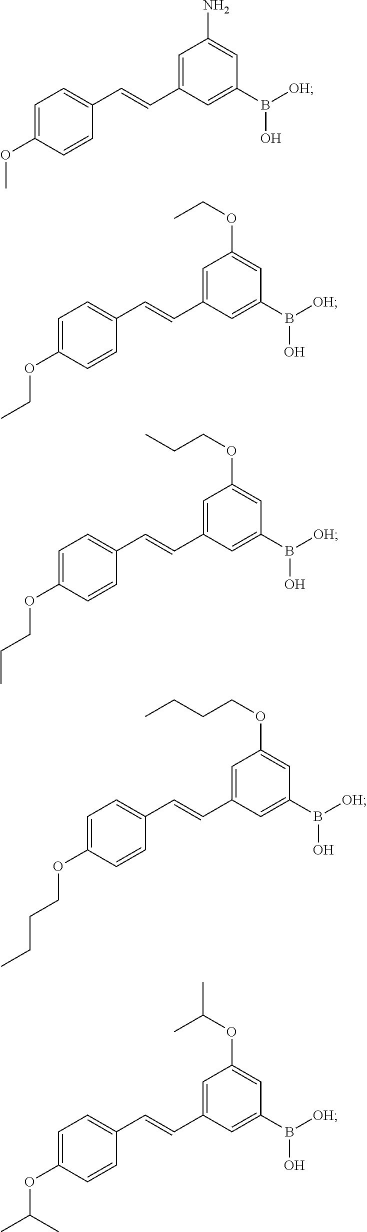 [SMILES: COc1ccc(/C=C/c2cc(N)cc(B(O)O)c2)cc1 ; CCOc1ccc(/C=C/c2cc(OCC)cc(B(O)O)c2)cc1 ; CCCOc1ccc(/C=C/c2cc(OCCC)cc(B(O)O)c2)cc1 ; CCCCOc1ccc(/C=C/c2cc(OCCCC)cc(B(O)O)c2)cc1 ; CC(C)Oc1ccc(/C=C/c2cc(OC(C)C)cc(B(O)O)c2)cc1]

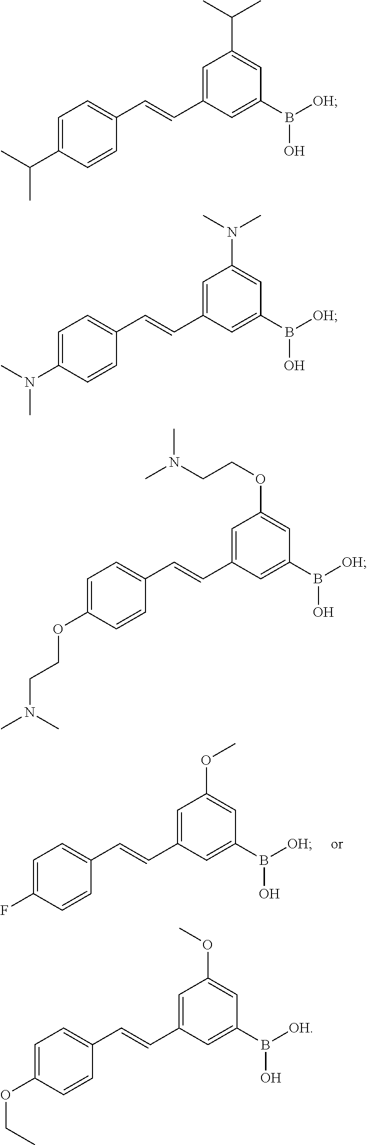 [SMILES: CC(C)c1ccc(/C=C/c2cc(B(O)O)cc(C(C)C)c2)cc1 ; CN(C)c1ccc(/C=C/c2cc(B(O)O)cc(N(C)C)c2)cc1 ; CN(C)CCOc1ccc(/C=C/c2cc(OCCN(C)C)cc(B(O)O)c2)cc1 ; COc1cc(/C=C/c2ccc(F)cc2)cc(B(O)O)c1 ; CCOc1ccc(/C=C/c2cc(OC)cc(B(O)O)c2)cc1]

6. A compound having a structure of Formula IV: 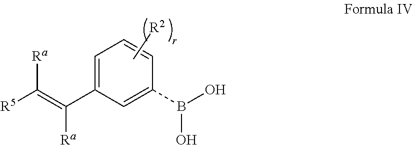
or a pharmaceutically acceptable salt, stereoisomer, tautomer, or prodrug thereof, wherein
each R^{a} independently is hydrogen, halogen, aliphatic, heteroaliphatic, haloaliphatic, aromatic, or any combination thereof;
R² is selected from aliphatic, haloaliphatic, halogen, —C(O)OR^{b}, —C(O)N(R^{b})₂, —C(O)H, —OC(O)R^{b}, —OC(O)OR^{b}, —NR^{b}S(O)₂R^{b}, —S(O)₂N(R^{b})₂, —[(C(R^{b})₂)ₙO]ₘR^{b}, —OR^{b}, —SR^{b}, —N(R^{b})₂, wherein each R^{b} independently is hydrogen, aliphatic, heteroaliphatic, haloaliphatic, aromatic, or combinations thereof, wherein each of n and m independently is an integer ranging from 1 to 50; and
R⁵ is either selected from naphthyl, pyridinyl, pyrrole, furanyl, thiophenyl, quinolinyl, piperidinyl, azepanyl, or diazabicyclooctanyl, or is an aromatic group comprising an R³ substituent, wherein the R³ group is selected from aliphatic, haloaliphatic, halogen, —C(O)OR^{b}, —C(O)N(R^{b})₂, —C(O)H, —OC(O)R^{b}, —OC(O)OR^{b}, —NR^{b}S(O)₂R^{b}, —S(O)₂N(R^{b})₂, —[(C(R^{b})₂)ₙO]ₘR^{b}, —OR^{b}, —SR^{b}, —N(R^{b})₂, wherein each R^{b} independently is hydrogen, aliphatic, heteroaliphatic, haloaliphatic, aromatic, or combinations thereof, wherein each of n and m independently is an integer ranging from 1 to 50; and
r is an integer selected from 0 or 1.

7. The compound of claim 6, wherein the compound further has a structure of Formula IVA

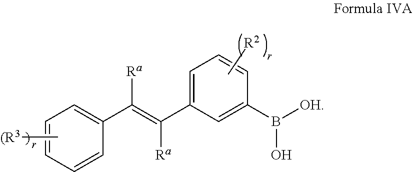

8. The compound of claim 6, wherein the compound further has a structure of Formulas IVB, IVC, IVD, IVE, or IVF

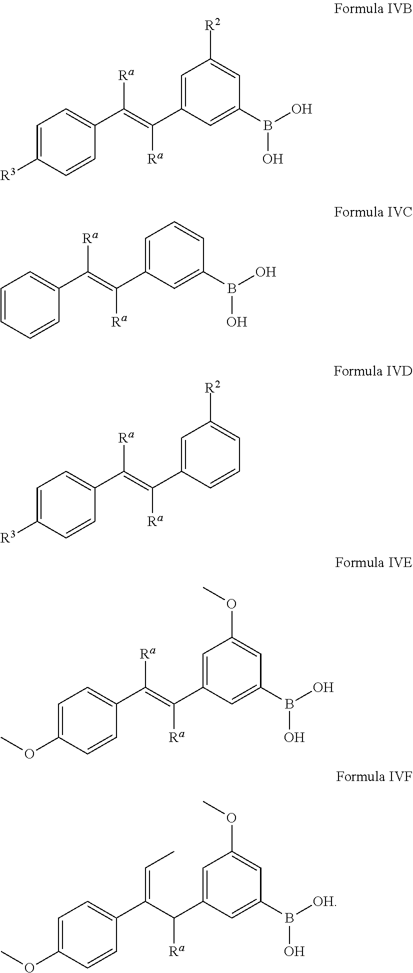

9-10. (canceled)

11. The compound of claim 6, wherein:
each R^{a} independently is hydrogen, F, Br, Cl, I, or alkyl;
each R^{a} independently is F, ethyl, phenyl, or -PhO(CH₂)₂NMe₂: or each R^{a} is different.

12-13. (canceled)

14. The compound of claim 6, wherein the compound is any one of:

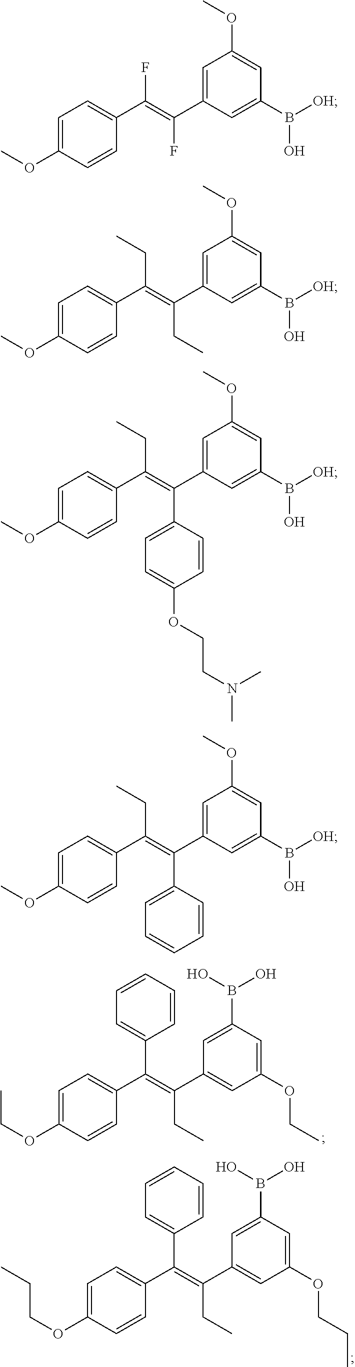

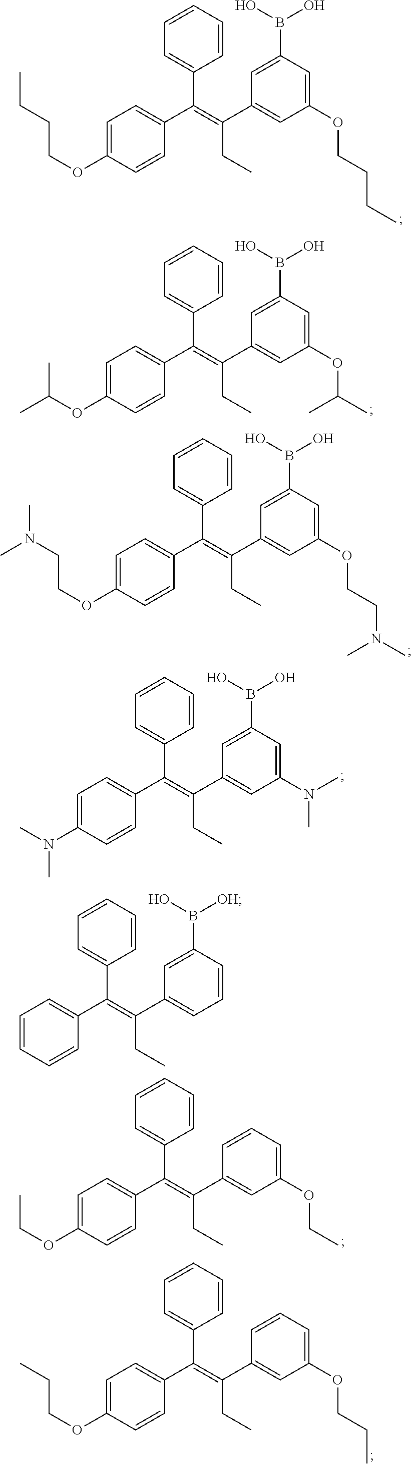

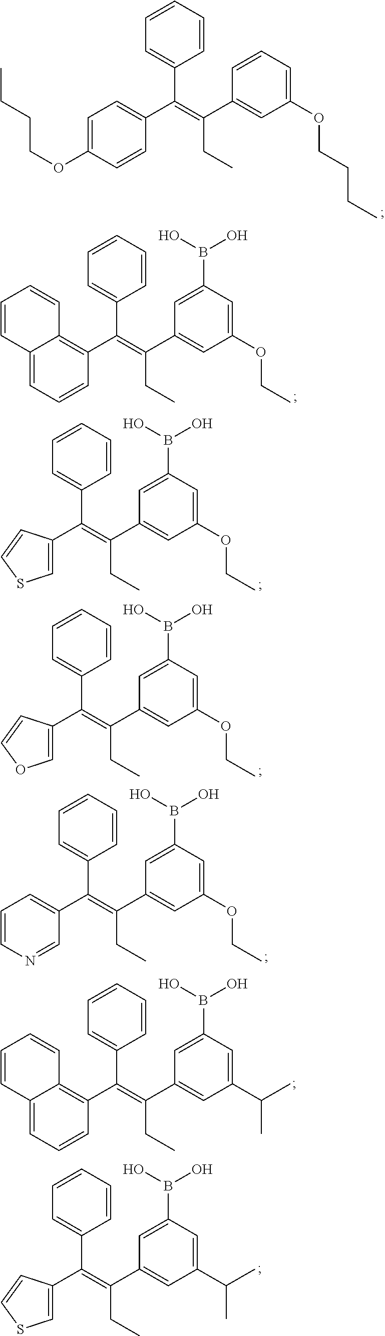

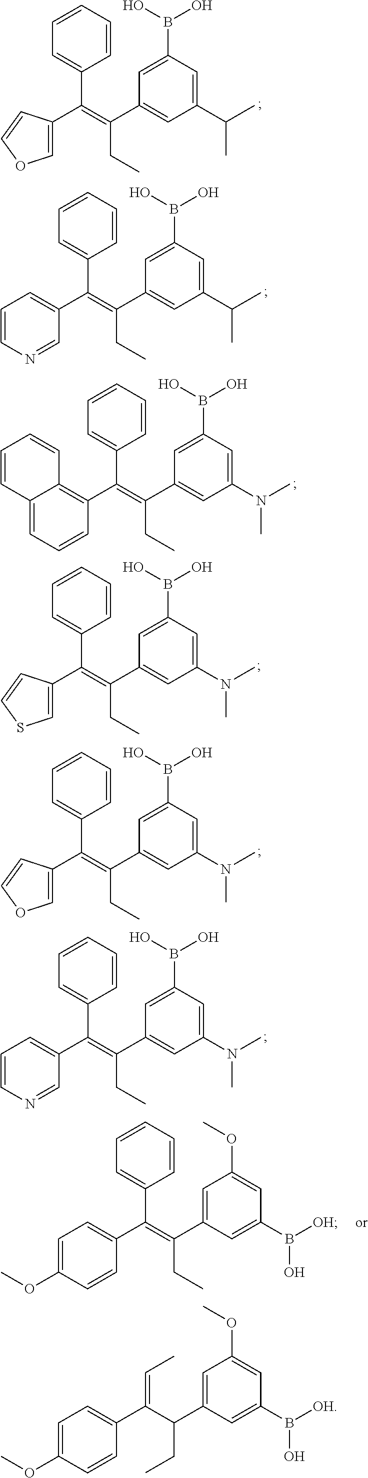

15. A compound having a structure of Formula III, IIIA, IIIB, or IIIC 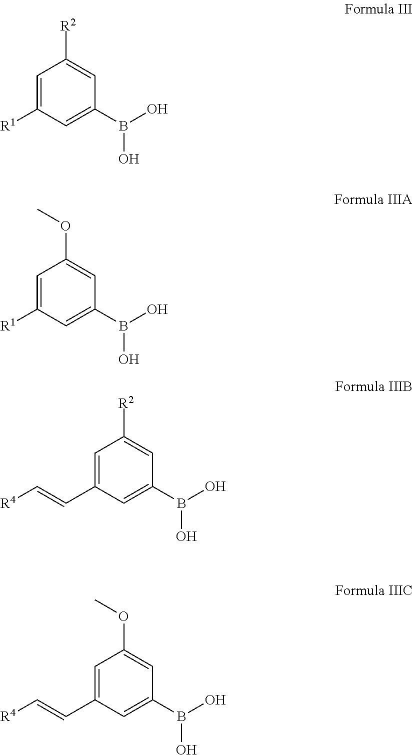
or a pharmaceutically acceptable salt, stereoisomer, tautomer, or prodrug thereof, wherein
R¹ is —[(CH═CH)-]ₚheterocyclic, —[(CH═CH)-]ₚaromatic, or —[(CH═CH)-]ₚheterocyclic-aromatic, wherein p is 0 or 1;
R² is selected from aliphatic, haloaliphatic, halogen, —C(O)OR^{b}, —C(O)N(R^{b})₂, —C(O)H, —OC(O)R^{b}, —OC(O)OR^{b}, —NR^{b}S(O)₂R^{b}, —S(O)₂N(R^{b})₂, —[(C(R^{b})₂)ₙO]ₘR^{b}, —OR^{b}, —SR^{b}, —N(R^{b})₂, wherein each R^{b} independently is hydrogen, aliphatic, heteroaliphatic, haloaliphatic, aromatic, or combinations thereof, wherein each of n and m independently is an integer ranging from 1 to 50; and
R⁴ is naphthyl, pyridinyl, pyrrole, furanyl, thiophenyl, quinolinyl, piperidinyl, azepanyl, or diazabicyclooctanyl.

16. The compound of claim 15, wherein R¹ is —[(CH═CH)-]ₚheterocyclic or —[(CH═CH)-]ₚheterocyclic-aromatic and wherein the heterocyclic group comprises 2 to 10 carbon atoms and one or more heteroatoms selected from oxygen, nitrogen, or combinations thereof.

17. The compound of claim 15, wherein R¹ is —[(CH═CH)-]ₚaromatic or —[(CH═CH)-]ₚheterocyclic-aromatic and the aromatic group is an aryl group or a heteroaryl group.

18. The compound of claim 17, wherein R¹ is —[(CH═CH)-]ₚheterocyclic-aromatic and the heterocyclic-aromatic group comprises one or more heterocyclic groups fused with one or more aromatic groups.

19. The compound of claim 15, wherein p is 0 and R¹ is carbazolyl, dihydrobenzodioxinyl, dibenzofuranyl, or xanthenyl.

20. The compound of claim 15, wherein p is 0, wherein the compound is any one of:

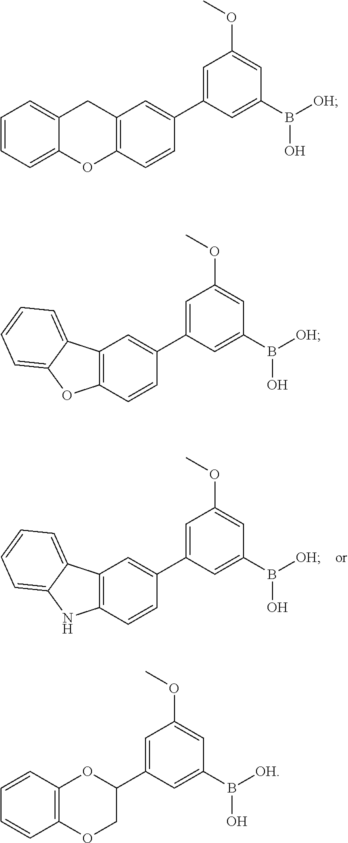 [SMILES: COc1cc(B(O)O)cc(-c2ccc3c(c2)Cc2ccccc2O3)c1 ; COc1cc(B(O)O)cc(-c2ccc3oc4ccccc4c3c2)c1 ; COc1cc(B(O)O)cc(-c2ccc3[nH]c4ccccc4c3c2)c1 ; COc1cc(B(O)O)cc(C2COc3ccccc3O2)c1]

21. The compound of claim 15, wherein R¹ is —[(CH═CH)-]ₚheterocyclic, —[(CH═CH)-]ₚaromatic, or —[(CH═CH)-]ₚheterocyclic-aromatic, wherein p is 1.

22. The compound of claim 15, wherein the compound has a structure of Formula IIIB or IIIC: 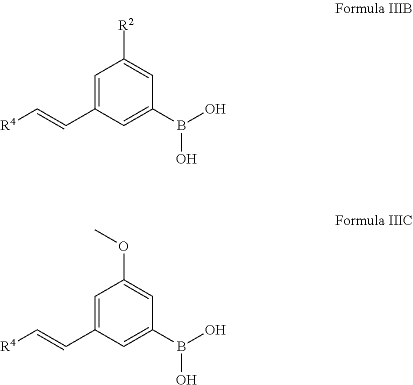
wherein
R² is isopropyl, —NMe₂, —OEt, —OPr, —OBu, —O(CH₂)₂N(Me)₂, or —OiPr; and
R⁴ is naphthyl, pyridinyl, pyrrole, furanyl, or thiophenyl

23. The compound of claim 22, wherein the compound is any one of:

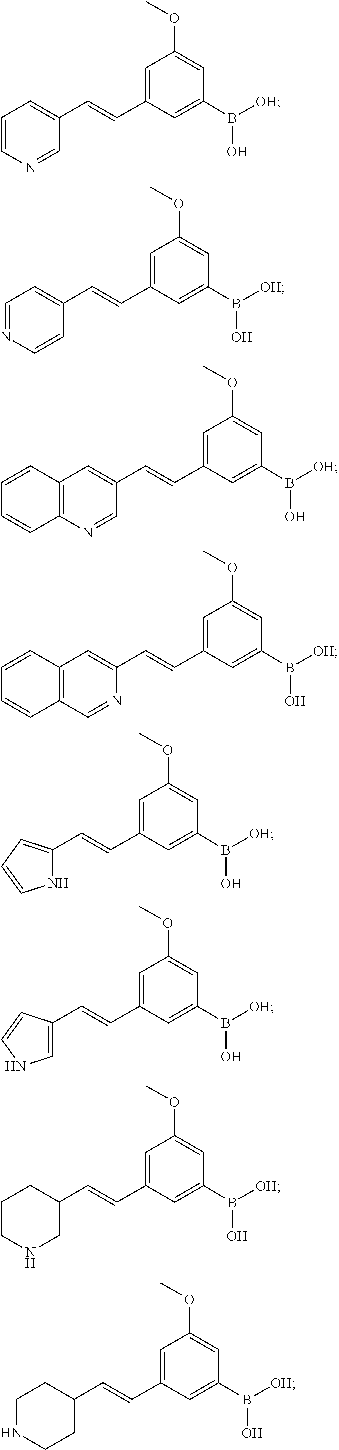

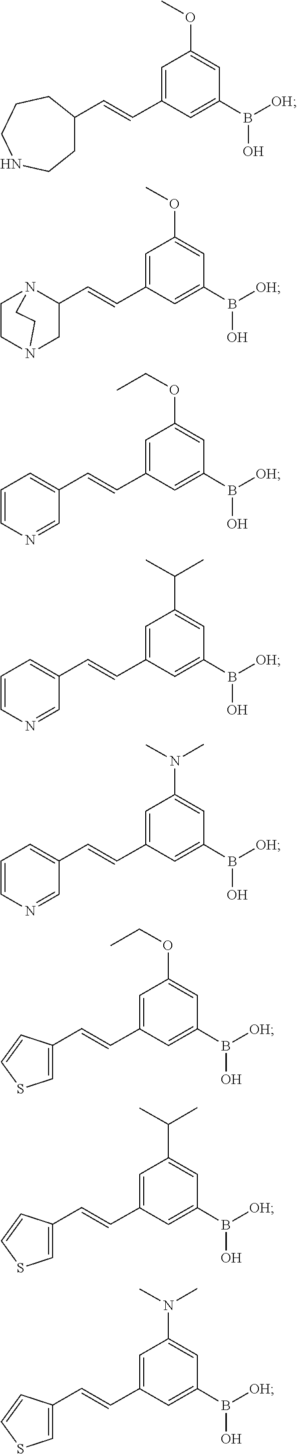

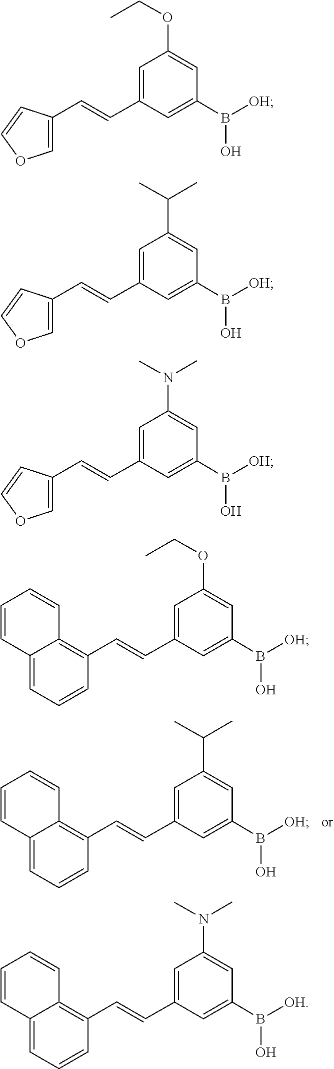

24. A composition comprising the compound of claim 1 and a pharmaceutically acceptable carrier.

25. A method of treating a subject with cancer or a neurodegenerative disease, comprising administering to the subject an effective amount of the compound of claim 1.

26-28. (canceled)

29. The method of claim 25, wherein the compound or composition is administered orally.

30. (canceled)

31. A method of treating triple-negative breast cancer, brain cancer, or a neurodegenerative disorder in a subject, comprising administering to the subject an effective amount of a composition comprising: 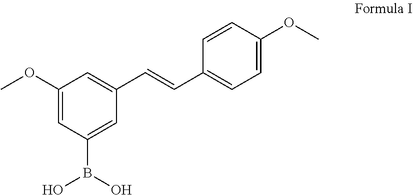
or a pharmaceutically acceptable salt, stereoisomer, tautomer, or prodrug thereof, thereby treating the triple-negative breast cancer, brain cancer, or neurodegenerative disorder.

32-33. (canceled)

34. The method of claim 31, wherein the compound is administered to the subject orally.

35. (canceled)
